(19) United States
(12) Patent Application Publication
(10) Pub. No.: US 2004/0092010 A1
(43) Pub. Date: May 13, 2004

(54) Method of proliferating and inducing brain stem cells to differentiate to neurons

(76) Inventors: Ariel Ruiz I Altaba, New York, NY (US); Arturo Alvarez-Buylla, San Francisco, CA (US); Daniel A. Lim, San Francisco, CA (US); Nadia Dahmane, Marseille (FR); Veronica Palma, New York, NY (US)

Correspondence Address: KLAUBER & JACKSON, 411 HACKENSACK AVENUE, HACKENSACK, NJ, 07601

(21) Appl. No.: 10/414,267

(22) Filed: Apr. 15, 2003

Related U.S. Application Data
(60) Provisional application No. 60/372,508, filed on Apr. 15, 2002.

(51) Int. Cl. C12N 5/06; C12N 5/08

(52) U.S. Cl. 435/354; 435/368

(57) ABSTRACT

The present invention discloses methods of producing neuronal cells from stem cells, particularly from adult brain stem cells. The use of such neuronal cells in the treatment and/or prevention of neurological diseases, conditions and/or injuries is also disclosed. In addition, the present invention provides a novel source of neuronal cells for use as a laboratory tool.

### CROSS-REFERENCE TO RELATED APPLICATIONS

The present application is a non-provisional application claiming the priority of provisional U.S. Serial No. 60/372,508 filed Apr. 15, 2002, the disclosure of which is hereby incorporated by reference in its entirety. Applicants claim the benefit of this application under 35 U.S.C. §119(e).

### GOVERNMENTAL SUPPORT

The research leading to the present invention was supported, at least in part, by grants from the National Institute of Neurological Disorders and Stroke, Grant No. R01 N537352 and from the National Cancer Institute, Grant No. R01 CA78736. Accordingly, the Government may have certain rights in the invention.

### FIELD OF THE INVENTION

The present invention relates to methods of producing neuronal cells from stem cells, particularly from adult brain stem cells. The present invention also relates to the use of such neuronal cells including as a laboratory tool and/or in the treatment and/or prevention of neurological diseases and/or injuries.

### BACKGROUND OF THE INVENTION

In nature, all of the cells and cell types of an individual adult mammal are derived from a single undifferentiated cell, a fertilized oocyte, i.e., the zygote. The zygote is termed “totipotent” since it also is the precursor of certain non-embryonic cells, such as the cells that contribute to the placenta. Next in developmental potential are the stem cells. Stem cells are defined as progenitor cells that (i) produce differentiated progeny and (ii) can also self-renew (Temple, Nature Reviews 2:513-520 (2000)). Self-renewal is the ability to divide and form at least one daughter cell that maintains the same developmental potential as the parent cell had.

Embryonic stem cells (ES cells) are termed “pluripotent”, since they can form all of the cell types derived from the embryo, but unlike the totipotent zygote, they cannot form non-embryonic cells. Adult stem cells are termed “multipotent”, and retain the ability to differentiate into the various cell types of a specific tissue type, i.e., hematopoietic stem cells are capable of differentiating into any cell type of the blood, and brain stem cells are able to differentiate into the different cell types of the brain. In addition, recent studies have suggested that adult stem cells may have further plasticity than originally thought since it has been reported that bone marrow derived stem cells could generate muscle cells under the right conditions (Temple, Nature Reviews 2:513-520 (2000)). The cells with the least developmental potential are fully differentiated adult cells which normally cannot be converted into another cell type and are thereby defined as “unipotent”.

Thus, some fully differentiated post-mitotic cells, such as mammalian neurons, are incapable of dividing. The inability of mammalian neuron cells to divide not only prevents self-healing of spinal cord and brain injuries but also adversely impacts a number of neurodegenerative diseases and disorders. Alzheimer's Disease, for example, is a progressive, degenerative disease of the brain which involves the destruction of neurons, resulting in almost complete memory loss and eventual death. Approximately 4 million Americans suffer from Alzheimer's disease, and the cost of caring for these victims is at least $100 billion per year. Moreover, as the baby boomers continue to mature, the percentage of the population having Alzheimer's Disease will dramatically increase, with approximately 14 million Americans being expected to have Alzheimer's Disease by the middle of this century.

Other neurodegenerative diseases include Parkinson's Disease and amyotrophic lateral sclerosis (ALS). In Parkinson's Disease, neurons in the brain deteriorate and are unable to produce the neurotransmitter, dopamine, which results in stiffness, tremors, slowness and poverty of movement, along with difficulty with balance and walking. ALS involves a progressive destruction of neurons in the brain which results in the brain becoming disconnected from the peripheral muscles of the body causing paralysis and eventually, death.

Yet another type of neurodegenerative disease is dementia. Dementia generally describes a loss of cognitive or intellectual function. Many conditions can cause dementia, including degenerative loss and damage to neurons in the brain. Diseases which can cause dementia include Parkinson's, Creutzfeldt-Jakob, Huntington's and Multi-Infarct or vascular disease. Dementia also can be caused by multiple strokes in the brain.

Efforts to treat nerodegenrative diseases include the use of drugs and surgical techniques. For example, the drugs “DONEPEZIL” and “TACRINE” have been developed to treat Alzheimer's Disease, but have met with only limited success since they only appear to temporarily relieve some of the symptoms. “RILUTEK”, an anti-glutamate, has been developed to treat victims suffering from ALS and is intended to prolong the life span of victims of ALS. Unfortunately “RILUTEK” appears to only delay the onset of ALS symptoms for a few months. Drugs have also been proposed to treat Parkinson's Disease. Particular examples of such drugs include Monoamine Oxidase B (MaoB) inhibitors, which are intended to prevent dopaminergic death of neuronal cells of Parkinson's patients. However, these drugs have met with only limited success, and recent research suggests that any observed benefit from the administration of MaoB inhibitors in Parkinson's patients may actually be due to effects other than prevention of dopaminergic nerve cell death.

Due to the limited success of drug treatments, efforts have been made to replace damaged or dead neurons in the brain with transplanted neuronal precursor cells. Indeed, transplantation of such cells into the adult mammalian brain offers promise for the treatment of neurodegenerative diseases and disorders (Lindvall, Trends in Neurosciences 14:376 (1991); Isacson and Deacon, Trends in Neurosciences 20:477 (1997); Martinez-Serran and Bjorklund, Trends in Neurosciences 20:530 (1997)). Indeed, a source of neurons or of cells that can be induced to form neurons is critical for developing such treatments for neural injuries and/or neurodegenerative diseases and disorders (Bjorklund and Lindvall Nat. Neurosci. 3:537-544 (2000)). However, heretofore no practical source of neurons or cells that can be induced to form neurons has been disclosed.

One potential source of cells that can be used to form neuronal cells are neural stem cells (NSCs) which have been broadly defined as multipotent, self-renewing progenitor cells (Anderson, Neuron 30:19-35 (2001)). Indeed, neurons, astrocytes, and oligodendrocytes are all generated from NSCs in the central nervous system (CNS), whereas neurons, Schwann cells, other neural crest derivatives (including smooth muscle cells) are generated from NSCs in the peripheral nervous system [Anderson, Neuron 30:19-35 (2001)].

Multipotent cells have been identified in several regions of the central nervous system and at several developmental stages (Gage et al., Ann. Rev. Neurosci. 18:159-92 (1995); Marvin and McKay, Semin. Cell. Biol. 3:401-11 (1992); Skoff, The Neuroscientist 2:335-44 (1996)). In addition, purified preparations of neuronal progenitor cells have been reported (U.S. Pat. No. 5,735,505 Issued May 19, 1998 and U.S. Pat. No. 6,251,669 Issued Jun. 26, 2001). More recently, progenitor cells have also been isolated and successfully propagated from human post-mortem tissues (Palmer et al., Nature 411:42-43 (2001)). However, several difficulties have arisen in identifying sources of dividing cells that generate neurons because neuronal progenitor cells frequently fail to express neuronal markers and because heterogeneous populations of cells (including neuronal and non-neuronal cells) generally arise.

Neoplastic cell lines and immortalized neuronal precursors have been used to provide relatively homogeneous populations of cells. Because these cells are rapidly dividing, they generally show a limited ability to fully differentiate into cells with a neuronal phenotype. For example, PC12 cells derived from a pheochromocytoma fail to differentiate or maintain a differentiated state in culture in the absence of nerve growth factor (NGF) (Green and Tischler, Advances in Cellular Neurobiology, S. Federoff and L. Hertz, eds. (Academic Press, New York), (1982)). Additionally, these cells are tumor-derived and have neoplastic characteristics.

Similarly, embryonal carcinoma cell lines have been differentiated in culture under special conditions. NT2 cells, derived from a teratocarcinoma, will differentiate in culture only following extended treatment with retinoic acid. The NT2 cells, however, differentiate into both neuronal and non-neuronal cell types. The resulting mixed culture must be treated with mitotic inhibitors and then the cells replated to remove the dividing non-neuronal cells and approach a relatively pure population of neuronal cells (U.S. Pat. No. 5,175,103, Issued Dec. 29, 1992). These relatively pure neuronal cells nonetheless are tumor-derived and have neoplastic characteristics.

Neurogenesis in the adult mammalian brain takes place in the striatal subventricular zone (SVZ) of the lateral ventricular walls of the forebrain and in the subgranular layer of the dentate gyrus of the hippocampus (Lois and Alvarez Buylla, Science 264:1145-1148 (1994); as reviewed in Temple and Alvarez-Buylla, Curr Opin Neurobiol 9:135-141 (1999)). In these areas or niches there is the persistence of conditions favorable for the existence of stem cells and the generation of new neurons from them. In particular, astrocytes (B cells) function as stem cells in the adult SVZ and generate transiently amplifying cells (C cells) that then differentiate into migrating neuroblasts (A cells) (Doetsch et al., Cell 97:703-716 (1999)). Neuroblasts, i.e., A cells, will then join the rostral migratory stream to reach their final destination in the olfactory bulb where they will terminally differentiate as intemeurons (Luskin, Neuron 11(1):173-89 (1993); Lois and Alvarez Buylla, Science 264:1145-1148 (1994)).

However, the mechanisms involved in the orderly production of new neurons from neural stem cells are not clear. During embryogenesis, several secreted signal carriers have been shown to particulate in brain development (Kilpatrick et al., Mol. Cell. Neurosci. 6:2-15 (1996); Temple and Qian, Neuron 15:249-252 (1995); Gritti et al., J. Neurosci. 16:1091-1100 (1996); Li et al., J. Neurosci. 18:8853-8862 (1998); Marbie et al., J. Neurosci. 19:7077-7088 (1999); and Li and LoTurco, Dev. Neurosci. 22:68-73 (2000)). One of these secreted signal carriers is sonic hedgehog (SHH). SHH is involved in different aspects of development of the early CNS, where it appears to play an important role in cellular differentiation and cell proliferation. For example, SHH is required for the differentiation of floor plate cells and ventral neurons in the early neural tube (Echelard et al., Cell 75:1417-1430 (1993); Krauss et al., Cell 75:1431-1444 (1993); Roelink et al., Cell 76:761-775 (1994); Ruiz i Altaba et al., Mol. Cell. Neurosci. 6:106-121 (1995)) and it is also involved in granule cell precursor proliferation in the cerebellum (Dahmane and Ruiz i Altaba, Development 126:3089-3100 (1999); Wallace, Curr Biol 9:445-448 (1999); Weschler-Reya and Scott, Neuron 22:103-114.(1999)). Similarly, SHH promotes the production of neuronal and oligodendroglial lineages in vitro (Kalyani et al., J. Neurosci. 18:7856-7868 (1998); Zhu et al., Dev. Biol. 215:118-129 (1999)); and in vivo (Pringle et al., Dev. Biol. 177:30-42 (1996); Poncet et al., Mech. Dev. 60:13-32 (1996); Orentas et al., Development 126:2419-2429 (1999); Rowitch et al., J Neurosci (1999), 19:8954-8965 (1999); Lu et al., (2000), Neuron 25:317-329).

In the striatal subventricular zone (SVZ) of post-natal and adult mouse brains, stem cell astrocytes give rise to committed neuronal precursors, which then produce neurons. As indicated above, SHH is secreted from precise locations and at defined periods in the embryonic CNS, and has been shown to play an important role in neurogenesis. However, heretofore, the role of SHH had been believed to be solely as a stimulus for committed neuronal precursor cell proliferation or as an inductive signal for embryonic neural tube precursors to differentiate as neurons (see e.g., Ericson et al., Cell 87:661-673 (1996)). For example, SHH and FGF8 or other factors are thought to induce dopaminergic neurons from the anterior neural tube (Ye et al., Cell, 93:755-766 (1998); Matsuura et al., J. Neurosci. 21:4326-4335 (2001)), but this occurs with rarity in vitro (Stull and Iacuitti, Exp. Neurol. 169:36-43 (2001)). Indeed, the factor(s) required for the orderly differentiation of adult stem cells into neurons has heretofore not been identified.

Therefore, there is a need to develop a practical source of neuronal cells. More particularly, there is a need to provide protocols for producing such neuronal cells from stem cells. In addition, there is a need to provide methods of treating neural injuries and diseases/disorders using the neuronal cells produced.

The citation of any reference herein should not be construed as an admission that such reference is available as “Prior Art” to the instant application.

### SUMMARY OF THE INVENTION

The present invention provides methods of proliferating and differentiating vertebrate cells. In one such embodiment, the vertebrate cell is an embryonic stem cell. In a preferred embodiment the vertebrate cell is a mammalian cell from the Central Nervous System (CNS). One such method comprises culturing the cell in the presence of an agent that stimulates the SHH-GLI pathway. Preferably the cell is induced to differentiate into a neuron. In a more preferred embodiment of this method, the agent is sonic hedgehog or an active fragment thereof. In another embodiment, the agent is sonic hedgehog or a fragment thereof used in combination with a growth factor. In a more preferred embodiment, the agent is sonic hedgehog or fragments thereof and the growth factor is Epidermal Growth Factor (EGF). In an alternative embodiment, the agent is Indian hedgehog (IHH). In yet another embodiment, the agent is desert hedgehog (DHH). In a further embodiment, the agent is Indian Hedgehog or desert hedgehog (DHH) or fragments thereof used in combination with a growth factor. In a further embodiment, the agent is Indian Hedgehog or desert hedgehog (DHH) or fragments thereof used in combination with EGF.

In one embodiment of the method of the invention, the mammalian cell is a brain stem cell. More preferably, the brain stem cell is an adult neural stem cell. In another such preferred embodiment, the brain stem cell is a perinatal neural stem cell. In still another such preferred embodiment, the brain stem cell is a post-natal neural stem cell. Even more preferably, the adult neural stem cell, perinatal neural stem cell or post-natal neural stem cell is a human adult neural stem cell, a human perinatal neural stem cell or a post-natal neural stem cell. In an alternative embodiment, the brain cell is a mouse subventricular stem cell.

The present invention further provides methods of generating a neuron from a brain stem cell. In a preferred embodiment, the brain cell is an adult neural stem cell, perinatal neural stem cell or post-natal neural stem cell. One such method comprises culturing the brain cell in the presence of an agent that stimulates the SHH-GLI pathway. In a preferred embodiment, the agent is sonic hedgehog or an active fragment thereof. Even more preferably the adult neural stem cell, perinatal neural stem cell or post-natal neural stem cell is a human adult neural stem cell, perinatal neural stem cell or post-natal neural stem cell. In still another embodiment, the brain stem cell is a mouse subventricular stem cell.

The present invention further provides methods for treating and/or preventing a neurologic or neurodegenerative disease, disorder or condition in a mammal. In one such embodiment, the method comprises transplanting a neuronal cell prepared by a method of the present invention into the brain of the mammal. In an alternative embodiment, the method comprises transplanting an expression vector that encodes sonic hedgehog or an active fragment thereof into the brain of the mammal. In yet another embodiment, the SHH protein or fragment thereof is inserted into the brain of the mammal. In yet another embodiment, pharmaceutical compositions containing SHH proteins or active fragments thereof or small organic molecules that increase expression of SHH protein are envisioned for treatment and/or prevention of neurological or neurodegenerative diseases, disorders or conditions. Methods of delivery of such pharmaceutical compositions includes oral, sublingual, buccal, intravenous, intramuscular, subcutaneous, intrathecal, intracranial or intraventricular delivery. Such pharmaceutical composition would contain appropriate carriers to enhance delivery to the site of injury. In a preferred embodiment the mammal is a human.

In a particular embodiment, the neurologic condition being treated is due to an injury. In a specific embodiment of this type the injury is a spinal cord injury. In another particular embodiment of the present invention, the neurologic condition is due to brain damage arising from trauma to the head or due to a stroke. The neurologic condition may also be syndromic or sporadic loss of stem cells, e.g., for example, in holoprosencephaly.

In an alternative embodiment, the neurologic condition being treated is due to a neurodegenerative disease. In a particular embodiment of this type the neurodegenerative disease is Alzheimer's disease. In another embodiment the neurodegenerative disease is Huntington's disease. In still another embodiment the neurodegenerative disease is Parkinson's Disease. In yet another embodiment the neurodegenerative disease is multiple sclerosis. In still another embodiment the neurodegenerative disease is amyotropic lateral sclerosis (ALS). In yet another embodiment the neurodegenerative disease is progressive supranuclear palsy. In still another embodiment the neurodegenerative disease is Creutzfeldt-Jakob Disease. In yet another embodiment the neurodegenerative disease is epilepsy. In still another embodiment the neurodegenerative disease is dementia. In yet another embodiment the neurodegenerative disease is schizophrenia.

The present invention further provides methods for enhancing the neuronal content of an adult mammalian brain. One such embodiment comprises transplanting a neuronal cell prepared by a method of the present invention into the brain of the mammal. In an alternative embodiment the method comprises transplanting an expression vector that encodes a hedgehog protein or an active fragment thereof into the brain of the mammal, e.g., sonic hedgehog. In yet another embodiment, the hedgehog protein or fragment thereof is inserted into the brain of the mammal. In a preferred embodiment the mammal is a human.

Accordingly, it is a principal object of the present invention to provide a source of neuronal cells for laboratory and/or medicinal use. A further object of the present invention is to provide a protocol for proliferating and differentiating brain stem cells. A still further object of the present invention is to provide a method of treating spinal cord injuries.

It is a further object of the present invention to provide methods of treating neurological disorders. It is a further object of the present invention to provide a method of treating or curing diseases such as Parkinson's disease, ALS and Alzheimer's disease.

It is a further object of the present invention to provide a method to enhance brain activity. It is a further object of the present invention to provide a method of treating brain tumors through the administration of inhibitors of the SHH-GLI pathway to an animal subject. It is still a further object of the present invention to provide a method of delivering/expressing a specific gene in the CNS and/or brain of an animal subject through the administration of neuronal cells obtained/grown by the methods of the present invention that have been genetically modified to encode the specific gene.

Other aspects and advantages will become apparent from a review of the ensuing detailed description taken in conjunction with the following illustrative drawings.

### BRIEF DESCRIPTION OF THE DRAWINGS

FIGS. 1A-1G show the localization of Gli1 and Shh gene expression in the adult SVZ. FIGS. 1A, 1D, and 1F, show that the expression of Shh mRNA is detected in the lateral wall of the lateral ventricles (LV; FIGS. 1A-1B). At higher magnifications, Shh expression is detected in SVZ cells (FIG. 1F). Arrows point to sites of expression unless otherwise noted. Ep: ependyma. FIGS. 1B, 1E, and 1G show the expression of Gli1 mRNA in the lateral wall of the lateral ventricle. Arrows point to sites of expression. At higher magnification, Gli1 expression is mostly detected in deep SVZ cells. FIG. 1C shows the control section which demonstrates the lack of hybridization with Shh antisense RNA probes in the 4^{th} ventricle (4V). All of the in situ hybridizations shown in FIGS. 1A-1G are on cross sections. In all cases dorsal is to the top.

FIGS. 2A-2B show the analyses of gene expression in sorted SVZ cells. FIG. 2A is the RT-PCR analyses of postnatal and adult cells. Postnatal whole SVZ is also shown here as control. FIG. 2B is the RT-PCR analyses of Shh expression in the SVZ and adjacent striatum from the same animal. Note that Shh is expressed in the adult SVZ but is not detected in either B or E sorted cells (FIG. 2A; see also Example 1 below). Analyses were carried out with (+) and without (−) reverse transcriptase to test for contaminating genomic DNA.

FIGS. 3A-3E show that SHH controls proliferation and neurogenesis in the SVZ. FIG. 3A shows the quantification of the effects of SHH on the proliferation of dissociated P5 SVZ cells plated on an astrocytic monolayer. BrdU incorporation was quantified by immunofluorescence. FIG. 3B shows the quantification of the effects of blocking anti-SHH monoclonal antibody (5E1) on the proliferation of P5 SVZ cells after dissociation and re-aggregation. Cell proliferation was measured by radioactive thymidine incorporation. FIG. 3C shows the quantification of the effect of SHH on neurogenesis in dissociated adult SVZ cells plated on an astrocytic monolayer. Generation of new neurons was measured by co-labeling with Tuj1, identifying neurons, and anti-BrdU antibodies, identifying cells that replicated after BrdU addition. Measurements were done after three or seven days in vitro (DIV).

FIGS. 4A-4E show the models for the action of SHH on SVZ lineages. FIG. 4A shows the proposed lineage of SVZ cells from stem cells (B cells) to transiently amplifying cells (C cells) that give rise to migrating neuroblasts (A) (from Doetsch et al., Cell 97:703-716 (1999)). FIG. 4B shows that SHH acts on stem cells inducing symmetrical divisions of B cells, which transiently accumulate and then give rise C and A cells. FIG. 4 shows that SHH acts on stem cells to increase number of symmetrical divisions and/or rate of B to C transition, which then give rise to A cells. FIG. 4D shows that SHH acts on stem cells inducing symmetrical, non-renewing divisions of C cells, which amplify and then give rise to A cells. FIG. 4E shows that SHH acts on transiently amplifying C cells to increase their number or to accelerate the production of A cells.

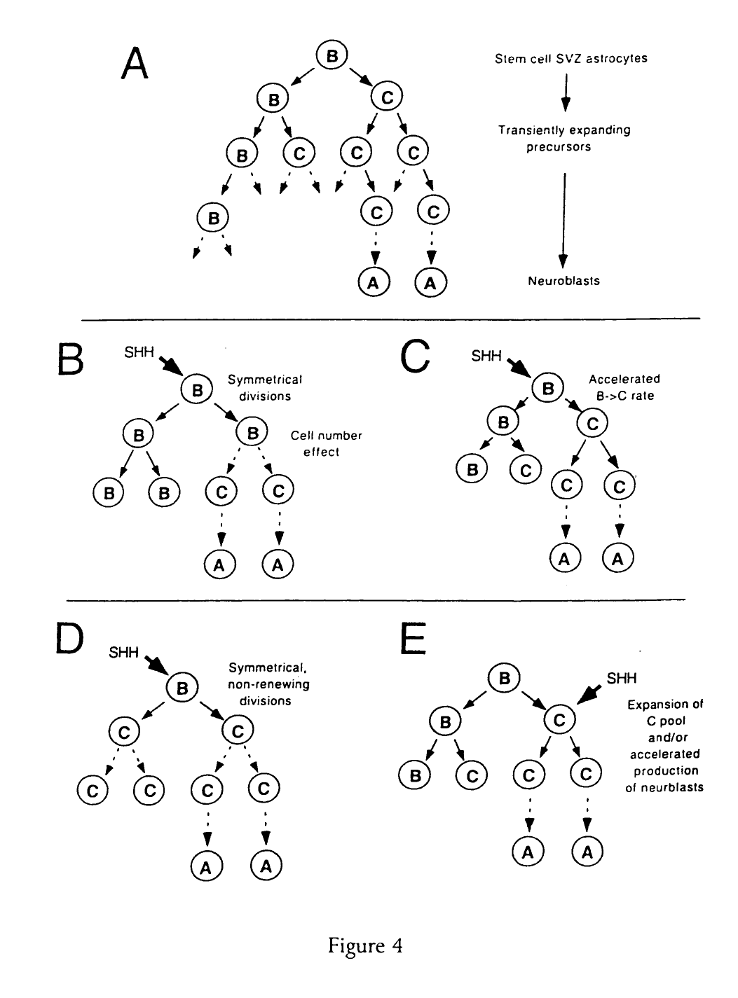

FIG. 5 shows the morphology and gene expression in the brains of Gli2 null animals. (A) Dorsal morphology of wild type (left) and Gli2−/− (right) dissected brains at E18.5. Anterior is to the top. (B, C) Comparison of lateral views of dissected cortex, and dorsal views of tectum and cerebellum in wild type (B) versus Gli2−/− (C) brains. Arrows point to the posterior cortex, tectum and cerebellum. D) Comparison of wild type and Gli2−/− cortices seen in parietal sagittal sections stained for hematoxilyn and eosin. E-I) BrdU incorporation in wild type (E, H) and in mutant (F, I) cortices, and quantification of cell proliferation (G). Shown is the mean number of BrdU⁺ cells per section±SEM from wild type and Gli2−/− animals. For simplicity, the vz was considered as the zone in between the ventricle and ˜5 cell diameters away, and the svz as that in between ˜5 and ˜10 cell diameters from the ventricle. P<0.05 comparing svz cells and P<0.01 for vz cell comparison. Note in (I) the uneven distribution of BrdU⁺ nuclei representing some variability in the thickness of the vz/svz. (J, K) Comparison of BrdU labeling in cerebellum of wild type (J) versus Gli2−/− in E18.5 samples (K). (L-Y) Images of in situ hybridization analyses of sagittal (L-U) hemisections from E18.5 and coronal sections from E15.5 (V-Y) of wild type and Gli2−/− animals probed with Gli1 (N, O), Gli2 (P, Q), Gli3 (R, S), NeuroD (L, M, T, U) clone 224 (V, W) or clone 53 (X, Y). Note the smaller hippocampus in (U, arrow) versus (T). (Z, ZZ) Quantification of the number of NeuroD⁺ (Z) and clone 53⁺ (ZZ) cells in the dorsal telencephalon. NeuroD⁺ (P<0.001) or clone 53⁺ (P<0.001). Cb: cerebellum; cp: cortical plate; Ctx: cortex; h: hippocampus; iz: intermediate zone; Med: medulla; St: striatum; svz: subventricular zone; Tct: tectum; vz: ventricular zone. Scale bar=800 μm for (A), 1.3 mm for (B,C), 75 μm for (D), 50 μm for (E,F,H,I), 130 μm for (J-M), 320 μm for N-U and 300 μm for (V-Y).

FIG. 6 shows the behavior of precursor and neurosphere-forming stem cells in Gli2 null brains. A-D) BrdU-positive cells in explant sections of wild type (A, C) and Gli2−/− (B, D) animals left untreated (A, B) or treated with SHH (C, D). E) Quantification of cell proliferation induced by SHH treatment in wild type and Gli2−/− cortical explants. Numbers represent cells per section±SEM, with n>10 sections of at least 3 independent explants in each condition. P<0.001 comparing Gli2−/− to wild type with or without SHH. (F) RT-PCR analysis of gene expression in untreated or SHH treated wild type versus Gli2−/− parietal cortical explants at E18.5. Expression of the housekeeping gene Hprt is used as internal control. Tbr1 expression confirms the cortical identity of the explants. A heterozygote Gli2+/− sample is used to show the Neo-containing and wt alleles. G-J) Phase contrast images of representative cortical nsp cultured from wild type (G, H) and Gli2−/− (I, J) animals at E18.5. K) RT-PCR analysis of cortical nsps. Note the loss of Gli1 expression, the shift in the Gli2 mutant allele band (arrows), the reduced Gli3 expression and the induction of Ihh and to a lesser extent of Dhh in Gli2−/− cells. Hprt is shown as a control. L-O) Expression of Nestin in precursors (L), of TuJ1 in neurons (M), of GFAP in astrocytes (N) and of O4 in oligodendrocytes (O) in Gli2−/− nsps. Nuclei were counterstained with DAPI. P, Q) Quantification of nsp size at E15.5 (P) and E18.5 (Q). The average of 20 nsp from 2 independent experiments is shown, P<0.05 for E15.5; P<0.001 for E18.5. R, S) Quantification of nsps obtained in cloning assays. One out of three independent experiments is shown for E15.5. P<0.001. Scale bar=300 μm for (A-D), 40 μm for (G), 75 μm for (H-J) and 10 μm for (L-O).

FIG. 7 shows precursor proliferation and neurosphere-forming cells in Shh null brains. A) Morphology of wild type (left, dorsal view) and Shh−/− (right, side view) E18.5 dissected brains. Anterior is to the top. B-E) Comparison of wild type (B, D) and Shh−/31 (C, E) cortical nsp, obtained at E15.5 (B, C) or E18.5 (D, E). F, G) BrdU incorporation assay on E18.5 attached nsps. H-K) Differentiation of Nestin positive nsp (H) into neurons (Tuj1, I), astrocytes (GFAP, J) and oligodendrocytes (O4, K) is not impaired in Shh null cultures. L). RT-PCR analysis of E18.5 wild type and Shh null nsp cultures. M) Quantification of wild type versus Shh−/− E15.5 and E18.5 nsp size in a single cell clonal dilution assay. E15.5 wild type, n=12; Shh null, n=11: E18.5 wild type, n=11; Shh null, n=14. P<0.001 for E15.5, P<0.05 for E18.5. N) Quantification of wild type versus Shh−/− E15.5 and E18.5 nsp number. P<0.001. O) Quantification of the number of BrdU+cells (4 days for E15.5, 1 week for E18.5) after a 7 h pulse in wild type versus Shh−/− nsp. P<0.001 for E15.5 and E18.5. Scale bar=620 μm for (A), 90 μm for (B,C,E), 70 μm for (D), 45 μm for (F,G) and 15 μm for (H-K).

FIG. 8 shows that in vivo treatment with cyclopamine inhibits neocortical proliferation and increases the number of neurosphere-forming cells. A-E) Characteristics of nsp-forming cells isolated at E17.5 from control and cyc treated embryos. The mean of 5 animals, processed independently, is shown. A) Quantification of the number of nsps formed in a cloning assay of primary culture and first passage cells. B) Quantification of nsp size. A minimum of n=10 nsp were selected to measure the nsp diameter of primary and first passage cultures. Note the difference in nsp number (for primary culture P=0.4 and for the first passage P<0.001) and size (for primary culture P=0.9 and for the first passage P<0.001) between control and cyc treated animals. C) Quantification of BrdU incorporation in primary cultures plated in the absence of growth factors. P<0.05. D, E) Phase contrast images of representative first passage nsp cultures from control (D) and in vivo cyc treated (E) animals. F) Proliferation response of plated nsps to different concentrations of EGF with (darker bars) or without (lighter bars) added SHH (5 nM) after 1 week. A 7 h pulse of BrdU was given. Shown is the total number of BrdU⁺ cells±SEM per well. Comparing to no SHH and decreasing concentrations of EGF: 5 nM P<0.5; 2.5 nM P<0.001; 0.5 nM P=0.001, 0.25 nM P<0.01 and 0.05 nM P<0.001. G) Quantification of BrdU⁺ cells in a 24 h cell culture assay in the presence of 1 ng/ml of EGF and varying concentrations of SHH (after a 7 h BrdU pulse). Compared to no SHH: 0.1 nM P=0.61; 0.5 nM P=0.58; 1 nM P<0.5; 5 nM P=0.013 and 25 nM P=0.596. Similar results were obtained with 48 h cultures. Scale bar=60 μm for (D,E).

FIG. 9 shows localization of Gli1 and Shh expression in the postnatal and adult SVZ. A,C) Expression of Shh mRNA in the lateral wall of the forebrain lateral ventricle (LV) of adult mice. At higher magnification, Shh expression is detected in SVZ cells (C). B, D, F-H) Expression of Gli1 mRNA in the lateral wall of the lateral ventricle of adult (B,D) and postnatal (P3; F-H) mice. E) Control section, showing lack of hybridization of Shh anti-sense RNA probes in tissue surrounding the 4^{th} ventricle (4V) of an adult mouse. All panels show cross sections. Arrows point to sites of expression. Dorsal is to the top. The significance of the expression in the ventral domain of the medial wall is unclear. I) Analyses of gene expression in sorted SVZ cells. RT-PCR analyses of postnatal (P5) and adult cells. Postnatal whole SVZ is also shown as control. J) RT-PCR analyes of Shh expression in the SVZ and adjacent striatum. Shh is expressed in the adult SVZ but it is not detected in either B or E sorted cells (panel I; see text). As control, all genes tested were expressed in dissected SVZ pieces. K) RT-PCR analyses of Shh, Gli and Ptch1 gene expression in P7 SVZ nsps. As controls, gene expression, including that of hprt, were measured in P7 brain RNA were tested with (+) or without (−) reverse transcriptase. Scale bar=350 μm for (A,B,F), 100 μm for (G), 200 μm for C-E) and 20 μm for (H).

FIG. 10 demonstrates that SHH regulates SVZ cell proliferation and neurogenesis. A) Quantification of the effects of SHH on the proliferation of dissociated P5 SVZ cells plated on an astrocytic monolayer as measured by BrdU incorporation (Lim et al., 2001). B) Quantification of the effects of blocking anti-SHH monoclonal antibody (5E1) on the proliferation of P5 SVZ cells after dissociation and reaggregation. Cell proliferation was measured by radioactive thymidine incorporation. C) Quantification of the effect of SHH on neurogenesis in dissociated adult SVZ cells plated on an astrocytic monolayer. Generation of new neurons was measured by co-labeling with Tuj1, identifying neurons, and anti-BrdU antibodies. Measurements were done after 3-7 days. D) Quantification of the effects of SHH on isolated P5, type A SVZ neuroblasts. Cells were sorted and grown in vitro on poly-lysine coated glass for 7 days with 10 nM SHH, passaged and cultured for 3 days and one week. Live neuroblasts were quantified by analysis of Tuj1 immunostaining and fluorescent nuclear counterstaining. The number of Tuj1-positive cells with non-pycnotic nuclei in SHH treated cultures were counted and expressed as a percentage of control cultures performed in parallel. E) Immunocytochemistry of one week SVZ culture on an astrocytic monolayer. BrdU was added to the culture medium 24 hours prior to fixation. Shown is the labeling of neurons with TuJ1 (red) and recently divided cells with anti-BrdU (green) antibodies. Note the large number of doubly labeled (yellow) cells representing newly born cells. F) Nomarski optics images of the sample panel shown in E). G, H) Postnatal SVZ cells were plated in medium with 10% FCS for 3 days, conditions allowing SVZ astrocytes to form a monolayer. The medium was then changed to serum-free medium, with or without SHH. After 4 more days, cultures were enzymatically dissociated to single cells, counted, and equal numbers of cells were plated nsp medium containing EGF (10 ng/ml). (G) 2.3-fold more nsps grew from SHH treated SVZ cells as compared to control SVZ cells. (H) Similarly, there were 2.4-fold more nsps derived from SHH-treated SVZ cells after passage of nsps from cultures in (G). Scale bar=45 μm for (E,F). In all cases, error bars show SEM of triplicate cultures.

FIG. 11 shows that in vivo blocking HH signaling decreases SVZ proliferation and increases the number of neurosphere-forming stem cells. A-F) Characterization of SVZ cells of HBC-vehicle control (A, B and E) and cyc (C, D and F) injected adults (after 7 days n cyc treatment in vivo). A,C) BrdU staining after a 2 h pulse). Arrows denote BrdU positive cells. Note that whereas some cyc-treated animals do show little or no BrdU staining (C, also detail in D), there is an intrinsic variability in the response of cyc-treated animals. I) Quantification of BrdU positive cells per section in control (n=6) and cyc-treated (n=11) adult mice. Four out of eleven animals did not respond to cyc treatment. Without counting these, the difference is greater (white bar). E, F) Detailed image of the lateral walls, showing cells positively stained for Nestin (green) and GFAP (red) of control (E) and cyc-treated animals (F). G, H, J and K) Characteristics of SVZ progenitor cells isolated at P9 from control and cyc treated pups (5 days treatment in vivo starting at P4). Two independent experiments, pooling 5 animals in each case for the nsp preparation, were done. G,H) Phase contrast images of representative nsps cultured from control (G) and cyc-in vivo treated (H) animals. J,K) Number and size of nsp, obtained in cloning assays, plated as described in FIG. 4. (size: primary culture P=0.2, and first passage P<0.001; number: primary culture P<0.5 and first passage P<0.05). LV: lateral ventricle. Scale bar=230 μm for (A,C), 50 μm for (B,D,E,F) and 130 μm for (G,H).

FIG. 12 shows a model for the action of SHH and EGF on amplifying precursors and stem cells in the brain. In vivo, slowly cycling stem cells (stem cell 1) give rise to transit amplifying precursors. These then give rise to committed precursors and to differentiated cells. In the SVZ this would correspond to the B->C->A->neuron lineage (Alvarez-Buylla et al., (2001) Nat. Rev. Neurosci. 2:287-293). In the developing neocortex, the distinction between stem cell and amplifying precursor is less clear. Stem cells and amplifying precursors can give rise to nsps (red dashed box) in vitro in the presence of EGF. Amplifying precursors form the bulk of the BrdU⁺ cells (green box). These can behave as stem cells (stem cell 2) and form the bulk of nsps formed in vitro (blue), as seen with SVZ C cells (Doetsch et al., (2002) Neuron 36: 1021-1034). SHH and EGF signaling act on amplifying precursors (black arrows) which can behave as stem cells, but also possibly on slow cycling stem cells (EGFR⁺/Gli1⁺ B cells in the SVZ)(gray arrows). Brain tumors may initiate from the inappropriate expansion of cells with stem cell properties (see Reya et al., (2001) Nature 414:105-111; Ruiz i Altaba et al., (2002b) Nat. Rev. Cancer 2:361-372) through enhanced SHH or EGF signaling acting on the stem cell 1 or, the amplifying precursor (stem cell 2) populations.

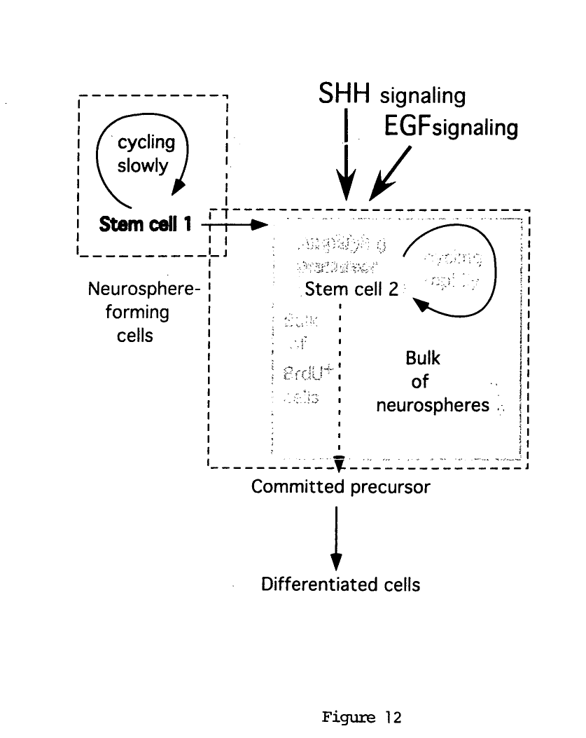

### DETAILED DESCRIPTION OF THE INVENTION

The present invention provides methodology for treating neurodegenerative diseases by providing methodology for producing large numbers of neurons. In addition, the present invention provides methodology for treating tumors, e.g., treating brain tumors.

More specifically the present invention discloses that sonic hedgehog (SHH) plays an important role in the perinatal, post-natal and adult brain by regulating cellular proliferation and neurogenesis in the striatal subventricular zone (SVZ). In situ hybridization analyses show that Shh and Gli1 are expressed in the adult SVZ. Analyses of gene expression in partially purified cells show that stem cells (SVZ astrocytes or B cells) appear to be the main target of SHH. In vitro analyses using dissociated SVZ cells demonstrate that SHH is sufficient to increase their proliferation, whereas blocking endogenous SHH signaling with a monoclonal antibody, cyclopamine, and/or a natural alkaloid results in decreased proliferation. SHH is thus an endogenous SVZ mitogen. SHH treatment also increases the number of neurons produced from stem cells. Together, these findings demonstrate that SHH is an important regulator of neurogenesis in the post-natal and adult mammalian brain. Moreover the present invention discloses that SHH or active fragments thereof can be used to increase neuronal cell proliferation, and that the administration of agents that block endogenous SHH signaling may be used in the treatment of tumors.

The present invention further provides methods of generating neuronal cells. The neuronal cells provided by the invention then can be transplanted into an adult mammalian brain following the culturing of adult brain stem cells in the presence of mature SHH or the recombinant N-terminal fragment named SHH-N. In one specific embodiment, 16-well glass culture slides are treated with 0.5 mg/ml poly-D-lysine (having a molecular weight of 300,000 or greater) using 2 μg/cm² fibronectin and 5 μg/cm² of laminin as substrates. Astrocytes are then plated at 50,000 per cm² in DMEM/10% (vol/vol) fetal calf serum. At confluence, astrocyte monolayers are rinsed with four changes of NB/B27. During culture, the NB/B27 medium is half changed every four days.

P5 SVZ cells are then plated on the astrocyte monolayer. Under these conditions SVZ precursors grow, as they normally do in vivo, and generate large colonies containing a majority of neurons. Addition of 5 ng/ml purified recombinant SHH, results in a significant increase in cell number and more importantly, the production of neuronal cells. The resulting cells can then be replated as above in the presence of SHH to generate additional neuronal cells. Repetition of this procedure can potentially result in a limitless supply of neuronal cells. Moreover, the methodology exemplified above can be readily scaled up to increase the absolute number of neurons obtained. For example, large numbers of stem cells can be treated with SHH or an agent that activates the SHH-GLI pathway.

Numerous neurologic or neurodegenerative diseases or disorders can be treated with the neuronal cells produced by the present invention including Alzheimer's disease, schizophrenia, Huntington's disease, Parkinson's Disease, multiple sclerosis, amyotropic lateral sclerosis (ALS), progressive supranuclear palsy, Creutzfeldt-Jakob Disease, epilepsy, and dementia. Furthermore, the methods of the invention have ready applications in treating brain damage in a mammal, resulting from a variety of reasons, including trauma to the head, and stroke. In addition, treating mental deficits associated with viable mutations affecting SHH signaling is also envisioned.

In addition, the neuronal cells can be used as a laboratory tool to identify factors involved in neural transmission, in drug assays, for transplant experiments, as a source of material for molecular screens for genes required to make a neuron as well as in the identification of factors that can bias neuronal fate.

Definitions

As used herein the “SHH-GLI pathway” is used interchangeably with the “Sonic hedgehog (Shh) signaling pathway” and is the signaling pathway initiated by a hedgehog protein binding to its receptor leading to the expression of a Gli protein. Factors involved and/or can function in the SHH-GLI pathway include any hedgehog protein such as sonic hedgehog, Indian hedgehog, and desert hedgehog, patched 1 and 2, smoothened, agonists and antagonists of such proteins, PKA, fused, suppressor of fused, costal-2, and modifiers and/or partners of any of the Gli 1, 2, or 3 proteins e.g., the Zic gene products.

As used herein the term “hedgehog” is used interchangeably with the term “HH” and is a cytokine that binds to the HH receptor to stimulate the beginning of the SHH-GLI pathway. The human SHH protein is encoded by the nucleotide sequence of SEQ ID NO:1 and has the amino acid sequence of SEQ ID NO:2. The murine SHH protein is encoded by the nucleotide sequence of SEQ ID NO:3 and has the amino acid sequence of SEQ ID NO:4. The rat SHH protein is encoded by the nucleotide sequence of SEQ ID NO:5 and has the amino acid sequence of SEQ ID NO:6. Xenopus HH protein is encoded by the nucleotide sequence of SEQ ID NO:7 and has the amino acid sequence of SEQ ID NO:8. The human Indian hedgehog (IHH) protein is encoded by the nucleotide sequences of SEQ ID NO:9 and/or 11 and has the amino acid sequence of SEQ ID NO:10 and/or 12. The murine desert hedgehog (DHH) protein is encoded by the nucleotide sequence of SEQ ID NO:13 and has the amino acid sequence of SEQ ID NO:14. Hedgehog proteins from species as different as humans and insects appear to play this same role and can be used interchangeably (see e.g., Pathi et al, (2001) Mech Dev. 106:107-117).

As used herein an “active fragment” of a hedgehog is a fragment of a hedgehog protein that can comprises the first 174 amino acids of the protein (not counting the signal sequence) and can stimulate both the in vitro proliferation and in vitro differentiation of a mouse subventricular stem cell such that the number of neuronal cells obtained in the presence of 100 μM or less active fragment of the HH is at least 2-fold greater than that obtained in its absence.

As used herein a mature neuronal cell is a cell having neuronal properties and characteristically showing dendrites or axons, expressing a marker protein such as nestin or exhibit electrical activity, recognized by an antibody such as Tuj1, or exhibits action potentials.

As used herein a perinatal neural stem cell is a neural stem cell isolated from an animal shortly before, during or shortly following the birth of the animal.

As used herein a “small organic molecule” is an organic compound (or organic compound complexed with an inorganic compound (e.g., metal)) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as green fluorescent protein (as described in U.S. Pat. No. 5,625,048 issued Apr. 29, 1997, and WO 97/26333, published Jul. 24, 1997, the disclosures of each are hereby incorporated by reference herein in their entireties) or luciferase.

A “vector” is a replicon, such as plasmid, phage, virus, or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

As used herein, the term “homologue” is used interchangeably with the term “ortholog” and refers to the relationship between proteins that have a common evolutionary origin and differ because they originate from different species. For example, mouse SHH is a homologue of human SHH.

As used herein the term “heterologous nucleotide sequence” is a nucleotide sequence that is added to a nucleotide sequence of the present invention by recombinant molecular biological methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by a nucleotide sequence of the present invention after the recombinant nucleic acid is expressed. In still another embodiment the heterologous nucleotide can function as a means of detecting a nucleotide sequence of the present invention. A heterologous nucleotide sequence can comprise non-coding sequences including restriction sites, regulatory sites, promoters and the like. Thus, the nucleic acids that encode the proteins being used and/or detected in the present invention can comprise a heterologous nucleotide sequence.

As used herein the terms “fusion protein” and “fusion peptide” are used interchangeably and encompass “chimeric proteins and/or chimeric peptides” and fusion “intein proteins/peptides”. A fusion protein of the present invention can comprise at least a portion of a HH protein of the present invention, for example, joined via a peptide bond to at least a portion of another protein or peptide including a second HH protein in a chimeric fusion protein.

As used herein a polypeptide or peptide “consisting essentially of” or that “consists essentially of” a specified amino acid sequence is a polypeptide or peptide that retains the general characteristics, e.g., activity of the polypeptide or peptide having the specified amino acid sequence and is otherwise identical to that protein in amino acid sequence except it consists ofplus or minus 10% or fewer, preferablyplus or minus 5% or fewer, and more preferably plus or minus 2.5% or fewer amino acid residues.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state govermnent or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host, i.e., a symptom of Parkinson's disease.

In a specific embodiment, the term “about” means within 20%, preferably within 10%, and more preferably within 5%.

Nucleic Acids Encoding SHH

The present invention contemplates use of nucleic acids encoding a Hedgehog family member such as sonic hedgehog (e.g., genomic or cDNA) and nucleic acids encoding active fragments thereof. HH can be used from any animal species, including insects, but preferably a mammalian source, and more preferably a human source. In accordance with the present invention there may be employed conventional molecular biology, microbiology, and recombinant DNA techniques within the skill of the art. Such techniques are explained fully in the literature. (See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); Sambrook and Russell, Molecular Cloning: A Laboratory Manual, Third Edition (2001) Cold Spring Harbor Laboratory Press, Cold Spring Harbor, N.Y.; DNA Cloning: A Practical Approach, Volumes I and II D. N. Glover ed. 1985; Oligonucleotide Synthesis, M. J. Gait ed. (1984); Nucleic Acid Hybridization, B. D. Hames & S. J. Higgins eds. (1985); Transcription And Translation, B. D. Hames & S. J. Higgins, eds. (1984); Animal Cell Culture. R. I. Freshney, ed. (1986); Immobilized Cells And Enzymes, IRL Press, (1986); B. Perbal, A Practical Guide To Molecular Cloning (1984); F. M. Ausubel et al. (eds.), Current Protocols in Molecular Biology, John Wiley & Sons, Inc. (1994)).

Accordingly, any animal cell potentially can serve as the nucleic acid source for the molecular cloning of an hh gene. The DNA may be obtained by standard procedures known in the art from cloned DNA (e.g., a DNA “library”), by chemical synthesis, by cDNA cloning, or by the cloning of genomic DNA, or fragments thereof, purified from the desired cell (see, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

The nucleotide sequence of the human SHH, SEQ ID NO:1, can also be used to search for highly homologous genes from other species, or for proteins having at least one homologous domain, using computer data bases containing either partial or full length nucleic acid sequences. Human ESTs, for example, can be searched. The human Shh sequence can be compared with other human sequences, e.g., in GenBank, using GCG software and the blast search program for example. Matches with highly homologous sequences or portions thereof can then be obtained.

If the sequence identified is an EST, the insert containing the EST can be obtained and then fully sequenced. The resulting sequence can then be used in place of, and/or in conjunction with SEQ ID NO:1 to identify other ESTs which contain coding regions of the SHH homologue (or SHH domain homologue). Plasmids containing the matched EST for example can be digested with restriction enzymes in order to release the cDNA inserts. If the plasmid does not contain the full length homologue the digests can be purified, e.g., run on an agarose gel and the bands corresponding to the inserts can be cut from the gel and purified. Such purified inserts are likely to contain overlapping regions which can be combined as templates of a PCR reaction using primers which are preferably located outside of the SHH open reading frame. Amplification should yield the expected product which can be ligated into a vector and used to transform an E coli derivative e.g., via TA cloning (Invitrogen) for example. A resulting full-length SHH homologue can be placed into an expression vector and the expressed recombinant SHH can then be assayed for its ability to stimulate the proliferation and differentiation of brain stem cells.

A modified HH can be made by altering nucleic acid sequences encoding the HH by making substitutions, additions or deletions that provide for functionally equivalent molecules. Preferably, such derivatives are made that have enhanced or increased effect on the proliferation and differentiation of adult brain stem cells.

Due to the degeneracy of nucleotide coding sequences, other DNA sequences which encode substantially the same amino acid sequence as an hh gene may be used in the practice of the present invention including those comprising conservative substitutions thereof. These include but are not limited to modified allelic genes, modified homologous genes from other species, and nucleotide sequences comprising all or portions of hh genes which are altered by the substitution of different codons that encode the same amino acid residue within the sequence, thus producing a silent change. Likewise, the HH derivative of the invention can include, but is not limited to, those containing, as a primary amino acid sequence, all or part of the amino acid sequence of an HH protein including altered sequences in which functionally equivalent amino acid residues are substituted for residues within the sequence resulting in a conservative amino acid substitution. And thus, such substitutions are defined as a conservative substitution.

For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to significantly affect apparent molecular weight as determined by polyacrylamide gel electrophoresis, or isoelectric point.

Particularly preferred conservative substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Amino acid substitutions may also be introduced to substitute an amino acid with a particularly preferable property. For example, a Cys may be introduced at a potential site for disulfide bridges with another Cys. Pro may be introduced because of its particularly planar structure, which induces β-turns in the protein's structure.

When comparing a particular full-length SHH for example, with human SHH having the amino acid sequence of SEQ ID NO:2, deletions or insertions that could otherwise alter the correspondence between the two amino acid sequences are taken into account. Preferably standard computer analysis is employed for the determination that is comparable, (or identical) to that determined with an Advanced Blast search at www.ncbi.nlm.nih.gov under the default filter conditions (e.g., using the GCG (Genetics Computer Group, Program Manual for the GCG Package, Version 7, Madison, Wis.) pileup program using the default parameters).

The genes encoding HH derivatives and analogs of the invention can be produced by various methods known in the art. The manipulations which result in their production can occur at the gene or protein level. For example, an hh gene sequence can be produced from a native hh clone by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of a nucleic acid encoding an HH, care should be taken to ensure that the modified gene remains within the same translational reading frame as the hh gene, uninterrupted by translational stop signals, in the gene region where the desired activity is encoded.

Additionally, the HH-encoding nucleic acid sequence can be produced by in vitro or in vivo mutations, to create and/or destroy translation, initiation, and/or termination sequences, or to create variations in coding regions and/or form new restriction endonuclease sites or destroy preexisting ones, to facilitate further in vitro modification. Preferably such mutations will further enhance the specific properties of the hh gene product identified to have the capabilities disclosed by the present invention. Any technique for mutagenesis known in the art can be used, including but not limited to, in vitro site-directed mutagenesis (Hutchinson, C., et al., J. Biol. Chem., 253:6551 (1978); Zoller and Smith, DNA, 3:479-488 (1984); Oliphant et al., Gene, 44:177 (1986); Hutchinson et al., Proc. Natl. Acad. Sci. U.S.A., 83:710 (1986)), use of TAB® linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70). A general method for site-specific incorporation of unnatural amino acids into proteins is described in Noren et al., (Science, 244:182-188 ( 1989)). This method may be used to create analogs with unnatural amino acids.

Expression of HH Polypeptides and Active Fragments Thereof

The nucleotide sequence coding for an HH, or a functionally equivalent derivative including a chimeric protein thereof, can be inserted into an appropriate expression vector, i.e., a vector which contains the necessary elements for the transcription and translation of the inserted protein-coding sequence. Such elements are termed herein a “promoter.” Thus, the nucleic acid encoding an HH is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding the corresponding HH and/or its flanking regions. Any person with skill in the art of molecular biology or protein chemistry, in view of the present disclosure, would readily know how to assay the HH expressed as described herein, to determine whether such a modified protein can indeed perform the functions of an HH taught by the present invention. Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. Expression of an SHH may be controlled by any promoter/enhancer element known in the art, e.g., a Simian Virus 40 (SV40) promoter, a cytomegalus virus promoter (CMV) promoter, or a tissue specific promoter such as the human glial fibrillary acidic protein promoter (GFAP) promoter, as long as these regulatory elements are functional in the host selected for expression. The resulting SHH protein or fragment thereof can be purified, if desired, by any methodology such as one that is well known in the art.

Production of Cells

Cells that can be used to produce the neuronal cells of the present invention can be obtained from stem cell lines and/or brain biopsies, including tumor biopsies, autopsies and from animal donors. Brain stem cells can then be isolated (concentrated) from non-stem cells based on specific “marker” proteins present on their surface such as nestin, or GFAP in specific cases e.g., for cells from the SVZ. In one such embodiment, a fluorescent antibody specific for such a marker can be used to isolate the stem cells using fluorescent cell sorting (FACS). In another embodiment an antibody affinity column can be employed. Alternatively, distinctive morphological characteristics can be employed. For example, stem cells residing in the ependymal layer, which lines the ventricles of the brain, can be identified by the presence of cilia.

Once the cells are isolated they can be proliferated and differentiated in the presence of SHH or any other factor (or molecule) that can activate the SHH-GLI pathway. Thus the cells can be cultured in vitro as described in the Example below, in the presence of SHH or any other factor (or molecule) that can activate the SHH-GLI pathway. Alternatively, the cells can be grown as neurospheres and then treated with a hedgehog protein such as SHH or any other factor (or molecule) that can activate the SHH-GLI pathway.

Transplantation of Neuronal Cells

The present invention extends to methods for treating neurodegenerative diseases or disorders, or brain damage in an adult mammal. Transplantation of the cells of the invention can be performed for the purpose of replacing damaged, degenerating or dead neuronal cells in an adult mammalian brain, delivering a biologically active molecule to a damaged, degenerating or dead neuronal cell to ameliorate a condition and/or to enhance existing neuronal cells. Thus, the methods of the present invention extend to treating an adult mammalian brain, including those predisposed to a disease or disorder and/or to enhancing brain function by increasing the neuronal content.

The neuronal cells provided by the invention can be transplanted into an adult mammalian brain following the culturing of adult brain stem cells in the presence of SHH as described herein. Transplantation of the neuronal cells can be achieved by stereotaxic injection of a cell suspension, and this injection can be in either a homotopic or heterotopic brain region. Transplantation can also be performed as described previously (see, Dunnett and Björklund eds., Transplantation: Neural Transplantation—A Practical Approach, Oxford University Press, Oxford (1992)). Cells of the invention can be suspended in a buffer solution, for example, or alternatively, whole tissue comprising cells of the invention can be transplanted. Dissociated cell suspensions can maximize cell dispersion and vascularization of the graft. Poor vascularization is a significant factor in poor graft survival. Cells can be labeled prior to transplant, if desired. Multiple transplants can be performed, depending upon the number of transplanted cells desired to be transplanted and the area of the target region that receives the transplanted cells.

Alternatively, cells can be initially washed and then suspended for transplantation in an equal volume of injectable isotonic solution comprising appropriate physiological osmolarity, which is substantially pyrogen and foreign protein free. An example of such an isotonic solution is isotonic saline. A pharmaceutically acceptable carrier can then be added to the cells forming a pharmaceutical composition.

The treatment methodology of the invention may be applied to a normal brain, more particularly to a normal brain predisposed through genetic or environmental factors to neurologic or neurodegenerative disease. Predisposition to any of the aforementioned diseases or conditions provides a basis for transplanting neuronal cells of the invention prior to the manifestation of the disease. Moreover, certain occupational risks of neurologic damage, such as exposure to industrial neurotoxins or to trauma, for example, in contact sports, may provide reason to prophylactically enhance the neuronal content of the brain by the methods of the invention. In addition, enhanced neurological function of the normal brain may be achieved by the methods of the invention, for both humans and non-human mammals, for the purpose of enhancing learning in special needs children for example, memory in senior citizens for example, and other brain functions.

In addition, the neuronal cells produced through the methods of the present invention can be modified to express specific genes (including heterologous genes) when the modified neuronal cells are transplanted in the CNS and/or recipient brain. For example, such recombinant cells may be used to deliver neurotrophins to surrounding cells.

Gene Therapy and Transgenic Vectors

A gene encoding a hedgehog protein, e.g., SHH, active fragment thereof, derivative thereof, or structural/functional domain thereof, can be introduced either in vivo, ex vivo, or in vitro in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. For example, in the treatment of neurological disorders or injuries, the striatal subventricular zone (SVZ) can be specifically targeted. Examples of particular vectors include, but are not limited to, a defective herpes virus 1 (HSV1) vector (Kaplitt et al., Molec. Cell. Neurosci., 2:320-330 (1991)), an attenuated adenovirus vector, such as the vector described by Stratford-Perricaudet et al. (J. Clin. Invest., 90:626-630 (1992)), and a defective adeno-associated virus vector (Samulski et al., J. Virol., 61:3096-3101 (1987); Samulski et al., J. Virol., 63:3822-3828 (1989)) including a defective adeno-associated virus vector with a tissue specific promoter, (see e.g., U.S. Pat. No. 6,040,172, Issued Mar. 21, 2000, the contents of which are hereby incorporated by reference in their entireties).

In a particular embodiment, for in vitro administration, an appropriate immunosuppressive treatment is employed in conjunction with the.viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nature Medicine, (1995)). In addition, it is advantageous to employ a viral vector that is engineered to express a minimal number of antigens.

In another embodiment the Shh gene can be introduced in a retroviral vector, e.g., as described in U.S. Pat. No. 5,399,346; Mann et al., (1983) Cell, 33:153; U.S. Pat. No. 4,650,764; U.S. Pat. No. 4,980,289; Markowitz et al., (1988) J. Virol., 62:1120; U.S. Pat. No. 5,124,263; International Patent Publication No. WO 95/07358, published Mar. 16, 1995; and Kuo et al., (1993) Blood, 82:845.

Targeted gene delivery is described in International Patent Publication WO 95/28494, published October 1995.

Alternatively, the vector can be introduced by lipofection. Liposomes may be used for encapsulation and transfection of nucleic acids in vitro. Synthetic cationic lipids designed to limit the difficulties and dangers encountered with liposome mediated transfection can be used to prepare liposomes for in vivo transfection of a gene encoding SHH (Felgner, et. al., Proc. Natl. Acad. Sci. U.S.A., 84:7413-7417 (1987); see Mackey, et al., Proc. Natl. Acad. Sci. U.S.A., 85:8027-8031 (1988)). The use of cationic lipids may promote encapsulation of negatively charged nucleic acids, and also promote fusion with negatively charged cell membranes (Felgner and Ringold, Science, 337:387-388 (1989)). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as the brain. Lipids may be chemically coupled to other molecules for the purpose of targeting (see Mackey et. al., Proc. Natl. Acad. Sci. U.S.A., 85:8027-8031 (1988)).

It is also possible to introduce the vector as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., (1992) J. Biol. Chem., 267:963-967; Wu and Wu, (1988) J. Biol. Chem., 263:14621-14624; Hartmut et al., Canadian Patent Application No. 2,012,311, filed Mar. 15, 1990).

In a preferred embodiment of the present invention, a gene therapy vector as described above employs a transcription control sequence operably associated with the nucleotide sequence encoding the SHH inserted in the vector. That is, a specific expression vector of the present invention can be used in gene therapy.

Such an expression vector is particularly useful to regulate expression of a therapeutic hh gene, e.g., sonic hedgehog gene. In one embodiment, the present invention contemplates constitutive expression of the hh gene, even if at low levels. Alternatively, a regulatable promoter may be used.

Administration

According to the present invention, a therapeutic composition, e.g., an SHH protein or active fragment thereof and a pharmaceutically acceptable carrier of the invention or an agent such as a small organic molecule that stimulates the SHH-GLI pathway and/or increases expression of SHH may be introduced parenterally, transmucosally, e.g., nasally. Preferably, administration is by intracranial, intrathecal or intraventricular administration. Alternatively, the therapeutic composition can be placed (e.g., injected) into the bloodstream after coupling the SHH protein or active fragment thereof to a carrier that will allow the SHH protein or active fragment thereof-carrier complex to cross the blood-brain barrier.

In a preferred aspect, an HH protein of the present invention can cross cellular or nuclear membranes, which would allow for intravenous or oral administration. Strategies are available for such crossing, including but not limited to, increasing the hydrophobic nature of a molecule; introducing the molecule as a conjugate to a carrier, such as a ligand to a specific receptor, targeted to a receptor; and the like.

The present invention also provides for conjugating targeting molecules to an HH protein. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. (On the other hand SHH may itself be considered a targeting molecule since it binds its own receptor). In a specific embodiment, the targeting molecule is an antibody. Preferably, the targeting molecule is a monoclonal antibody. In one embodiment, to facilitate crosslinking the antibody can be reduced to two heavy and light chain heterodimers, or the F(ab′)₂ fragment can be reduced, and crosslinked to the SHH protein via a reduced sulfhydryl.

Antibodies for use as targeting molecule are specific for cell surface antigen. In one embodiment, the antigen is a receptor. For example, an antibody specific for a receptor on a brain stem cell can be used. This invention further provides for the use of other targeting molecules, such as lectins, carbohydrates, proteins and steroids.

In another embodiment, the therapeutic compound can be delivered in a vesicle, in particular a liposome (see Langer, (1990) Science, 249:1527-1533; Treat et al., (1989) in Liposomes in the Therapy of Infectious Disease and Cancer, Lopez-Berestein and Fidler (eds.), Liss: New York, pp. 353-365; Lopez-Berestein, ibid., pp. 317-327; see generally ibid.).

In yet another embodiment, the therapeutic compound can be delivered in a controlled release system. For example, the polypeptide may be administered using intravenous infusion, an implantable osmotic pump, a transdermal patch, liposomes, or other modes of administration. In one embodiment, a pump may be used (see Langer (1990) supra; Sefton, (1987) CRC Crit. Ref. Biomed. Eng., 14:201; Buchwald et al., (1980) Surgery, 88:507; Saudek et al., (1989) N. Engl. J. Med., 321:574). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, (1983) J. Macromol. Sci. Rev. Macromol. Chem., 23:61; see also Levy et al., (1985) Science, 228:190; During et al., (1989) Ann. Neurol., 25:351; Howard et al., (1989) J. Neurosurg., 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity of the adult brain stem cells, e.g., the striatal subventricular zone (SVZ). Other controlled release systems are discussed in the review by Langer (1990) supra.

Pharmaceutical Compositions

In yet another aspect of the present invention, provided are pharmaceutical compositions of the above. Such pharmaceutical compositions may be for administration for nasal or other forms of administration. In general, comprehended by the invention are pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990), Mack Publishing Co., Easton, Pa. 18042 pages 1435-1712 which are herein incorporated by reference. The compositions may be prepared in liquid form, or may be in dried powder, such as lyophilized form.

Nasal Delivery

Nasal delivery of an HH protein or derivative thereof is also contemplated. Nasal delivery allows the passage of the protein to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Liquid Aerosol Formulations

The present invention provides aerosol formulations and dosage forms. In general such dosage forms contain a pharmaceutical composition of the present invention in a pharmaceutically acceptable diluent. Pharmaceutically acceptable diluents include but are not limited to sterile water, saline, buffered saline, dextrose solution, and the like.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

The formulations of the present embodiment may also include other agents useful for pH maintenance, solution stabilization, or for the regulation of osmotic pressure.

Aerosol Dry Powder Formulations

It is also contemplated that the present aerosol formulation can be prepared as a dry powder formulation comprising a finely divided powder form of pharmaceutical composition of the present invention and a dispersant. Formulations for dispensing from a powder inhaler device will comprise a finely divided dry powder containing pharmaceutical composition of the present invention (or derivative) and may also include a bulking agent, such as lactose, sorbitol, sucrose, or mannitol in amounts which facilitate dispersal of the powder from the device, e.g., 50 to 90% by weight of the formulation. The pharmaceutical composition of the present invention (or derivative) should most advantageously be prepared in particulate form with an average particle size of less than 10 mm (or microns), most preferably 0.5 to 5 mm, for most effective delivery to the distal lung.

In a further aspect, recombinant cells that have been transformed with an hh gene, e.g., sonic hedgehog gene and that express high levels of the polypeptide can be transplanted in a subject in need of the HH protein. Preferably autologous cells transformed with HH protein are transplanted to avoid rejection; alternatively, technology is available to shield non-autologous cells that produce soluble factors within a polymer matrix that prevents immune recognition and rejection.

Methods of Treatment, Methods of Preparing a Medicament

In yet another aspect of the present invention, methods of treatment and manufacture of a medicament are provided. Conditions alleviated or modulated by the administration of the present derivatives are those indicated above.

Dosages. For all of the above molecules, as further studies are conducted, information will emerge regarding appropriate dosage levels for treatment of various conditions in various patients, and the ordinary skilled worker, considering the therapeutic context, age and general health of the recipient, will be able to ascertain proper dosing.

A subject in whom administration of HH is an effective therapeutic regiment is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Specific Embodiments

The restricted nature of neurogenesis in the adult brain offers an opportunity for the identification of the molecular signals involved in the creation of a neurogenic niche. Recent work has shown that, unlike in the post-natal cortex (Li and Loturco, (2000) Dev Neurosci 22:68-73), bone morphogenetic protein (BMP) signaling inhibits neurogenesis and induces glial differentiation in the adult brain SVZ (Lim et al., (2000) Neuron 28:713-726 ). Ependymal cells, which cover the SVZ secrete the BMP antagonist Noggin, thereby creating a microenvironment in which neurogenesis can occur (Lim et al., (2000) Neuron 28:713-726). However, inhibition of BMP signaling by ectopic expression of Noggin does not induce neurogenesis in areas of the adult brain in which new neurons do not normally form, suggesting that several factors may normally be required to induce neurogenesis. Heretofore, the factor(s) required to induce neurogenesis has not been identified.

The involvement of Noggin and BMPs in adult neurogenesis suggests a parallel role of these factors in the development of the embryonic neural tube and the adult SVZ. In this context, it was hypothesized that the Sonic hedgehog (Shh)-Gli signaling pathway could be involved in the regulation of cell proliferation and neurogenesis in the SVZ of post-natal and adult mice, as it is involved in both cell type differentiation and proliferation in the earlier embryonic neural tube (reviewed in Ruiz i Altaba, (1999) Development 126:3205-3216 ).

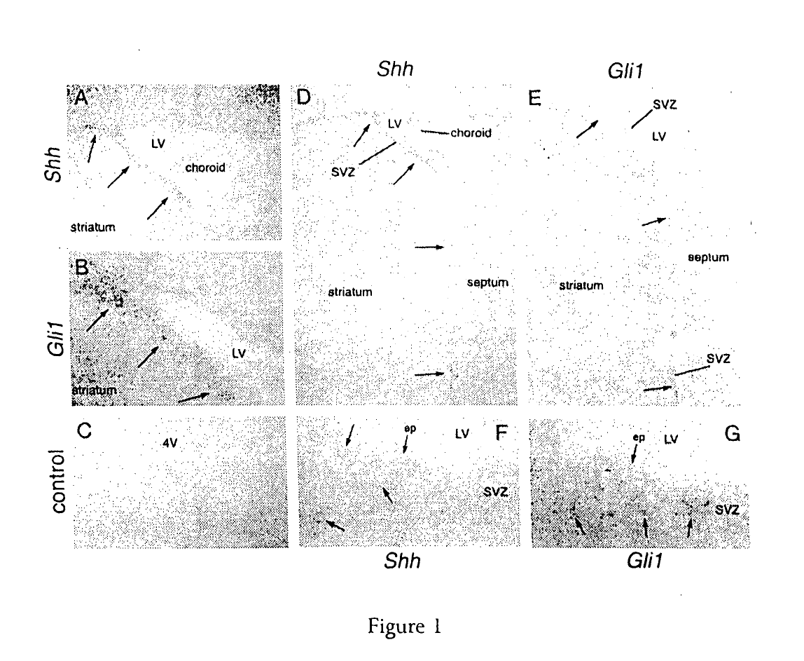

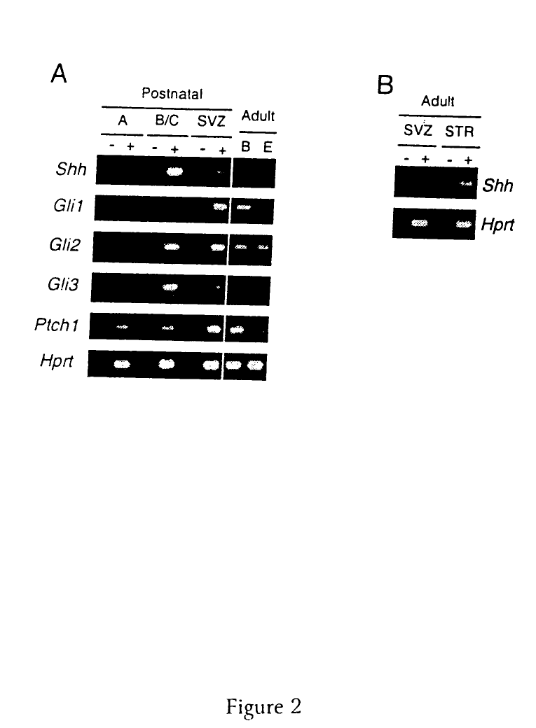

Localization of the expression of Shh and Gli1, a gene that is consistently induced by Shh signaling, was first performed by in situ hybridization on frozen sections (Dahmane et al., (1997) Nature 389:876-881). In the adult brain Shh is weakly but specifically expressed in the striatal SVZ (FIG. 1A, D). Its expression in the striatum (see FIG. 2B) adjacent to the SVZ was undetectable (FIG. 1D). Like with Shh, the expression of Gli1 was found in the SVZ (FIGS. 1B, 1E), but also in clusters of cells in the adjacent regions (FIG. 1E). The wider expression of Gli1 versus Shh could indicate the action of SHH at a distance from producing cells or the migration of responding cells. Expression of these genes in cells close to the edge of tissues was not detected in the walls of the 4^{th} ventricle, suggesting the specificity of the signal (FIG. 1C). Sense probe controls gave no signal.

Within the SVZ the expression of Shh was found throughout its thickness including cells closest to the ventricle, which may be ependymal cells (FIG. 1F). In contrast, expression of Gli1 was detected at higher levels in the deeper SVZ cells as compared with the more superficial cells (FIG. 1G). Thus, Gli1-expressing, SHH-responding cells are probably not located in the ependymal layer. Given the very low levels of expression of Shh in the SVZ, postnatal (Lim et al., (1999) Proc Natl Acad Sci USA 96:7526-7531) and adult SVZ cells were dissociated and sorted to perform RT-PCR assays testing for the expression of genes involved in SHH signaling, in order to better understand which cells in the SVZ express Shh and which may respond to it. In addition to Gli1 and Shh, the tested genes included the other two Gli genes, Gli2 and Gli3 and the gene encoding the SHH receptor Ptch1. Like Gli1, Ptch1 transcription is SHH-responsive (Goodrich et al., (1996) Genes Dev 10:301-312). In contrast, Gli2 can be activated by SHH in some contexts, whereas there appears to be an antagonistic relationship between SHH/Gli1 and Gli3 (e.g. Ruiz i Altaba, (1998) Genes Dev 10:301-312). In the postnatal SVZ, A cells only expressed low level of Gli2 and Ptch1 but not Shh or Gli1, whereas the fraction containing B and C cells expressed high levels of Shh, Gli2 and Gli3 but also Ptchl and very low levels of Gli1 (FIG. 2A). As a control, all genes were expressed in dissected but noncell-sorted SVZ pieces. In the adult SVZ, E cells expressed only low levels of Gli2 and Ptch1, whereas B cells expressed high levels of Gli1, Gli2 and Ptch1. The cDNAs from adult A cells were found not to be representative and were not used. Expression of Gli3 in adult B or E cells was not detected (FIG. 2A). As a control for RNA recovery, the levels of the housekeeping gene Hprt were always measured and all samples were tested with (+) or without (−) reverse transcriptase to eliminate any possible signal from contaminating genomic DNA.

Since SHH acts through Gli1/2 in other contexts (e.g., Lee et al., (1997) Development 124:2537-2552; Ruiz i Altaba (1998) Genes Dev 10:301-312), and both Gli1 and Ptch1 are reliably expressed in cells receiving the SHH signal (e.g., Goodrich et al., (1996) Genes Dev 10:301-312; Lee et al., (1997) Development 124:2537-2552; Hynes et al., (1997) Neuron 19:15-26), it was possible to derive functional relationships from the data disclosed herein. In post-natal animals, the B/C cells may both express the signaling molecule and respond to it. In adults, B cells seem to respond to SHH signaling but apparently do not produce SHH themselves. The lack of Shh expression in adult isolated B or E cells suggests that either other SVZ cells express it or that rare messages can be lost in the cell sorting and cDNA amplification procedure, as Shh is indeed expressed in the adult SVZ (FIG. 2B). Together, these data indicate that in post-natal mice B/C cells produce and respond to SHH. In addition, SHH may affect A cells, as these express Gli2 and Ptch1. In adults, the cell-sorting and cDNA amplification protocols were not sufficient to identify the cells expressing Shh. Nevertheless, as in postnatal-animals, the main target of SHH signaling is B cells. SHH could also have an effect on E cells as these express both Gli2 and Ptch1. The main conclusion that can be drawn from these expression profiles is that B cells are the main SHH target, which implies that stem cells respond to SHH.

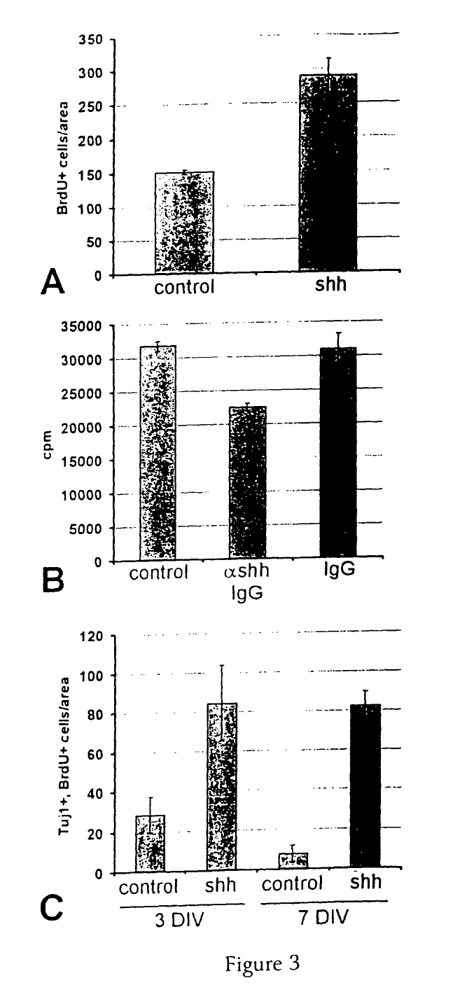

To test the role of SHH in the SVZ, dissociated P5 SVZ cells were plated on an astrocytic monolayer as previously described (Lim et al. (1999) Proc Natl Acad Sci USA 96:7526-7531; Lim et al., (2000) Neuron 28:713-726). Under these conditions SVZ precursors grow, as they normally do in vivo, and generate large colonies containing a majority of neurons. Addition of 5 ng/ml purified recombinant N-SHH, a dose previously shown to induce proliferation of cerebellar granule cell precursors (Dahmane and Ruiz i Altaba, (1999) Development 126:3089-3100) resulted in a 2-fold increase in the number of BrdU⁺ cells (FIG. 3A). The requirement of SHH for SVZ cell proliferation was tested by making aggregates of dissociated post-natal SVZ cells in the absence of the astrocytic monolayer, and treating them with anti-SHH monoclonal antibody (4 μg/ml 5E1 monoclonal antibody; Ericson et al. (1996) Cell 87:661-673) in the presence of tritiated thymidine. Addition of anti-SHH antibody decreased DNA replication by ˜30% as compared to sibling cultures treated with an isotype-matched unrelated antibody (FIG. 2B). These results indicate that SHH regulates the proliferation of SVZ cells and suggest that SHH is an endogenous SVZ mitogen.

To test if the proliferative effects of SHH could result in an increased number of neurons produced, dissociated adult SVZ cells were plated on an astrocytic monolayer and cultured in the presence or absence of SHH for 3 or 7 days in vitro (DIV; FIG. 3C). Addition of Shh increased the number of newly born Tuj1⁺ neurons by 3-fold after 3 DIV and by 10-fold after 10 DIV. The difference could reflect both a cumulative effect on neurogenesis as well as a decrease viability of neuron-generating stem cells in vitro in the untreated control samples. Nevertheless, these findings indicate that SHH has powerful effects on the proliferation of stem cells and the differentiation of neuronal precursors in the SVZ.

The findings further indicate that SHH acts as a mitogen of neuronal precursors, probably SVZ stem cell astrocytes (GFAP⁺/Gli1⁺/Gli2⁺/Ptch1⁺ B cells). These results are more difficult to reconcile with the suggested role of adult ependymal cells as stem cells (Johansson et al., (1999) Cell 96:25-34) since these do not express Gli1, although the fact that they express Gli2 and Ptch1 indicates that they may also respond to SHH. The role of SHH in ependymal cells, however, remains to be elucidated. The effects of SHH on the proliferation of SVZ cells are similar to those on cerebral cortical precursors where SHH is a mitogen for Nestin⁺ cells. The decision of neural stem cells to proliferate or differentiate into specific cell types probably depends on other extrinsic factors. For example, it is likely that BMPs and their inhibitors regulate the neurogenic pathway. Neurogenesis in the postnatal and adult SVZ could depend on the concerted action of SHH as disclosed herein and Noggin (Lim et al., (2000) Neuron 28:713-726). In addition, other signals like EGF (Tropepe et al., (1997) J Neurosci. 17:7850-7859, FGF (Qian et al., (1997) J Neurosci 17:7850-7859) and EphB2 (Conover et al., (2000) Nat Neurosci 3:1091-1097) are also probably involved. How these factors interact to regulate the production of different cell types in appropriate numbers remains unclear.

One possibility is that Gli proteins integrate different signaling inputs, such as FGFs and HHs (e.g., Lee et al., (1997) Development 124:2537-2552; Brewster et al., (2000) Development 127:4395-4405), and that SHH enhances proliferation of stem cells whereas Noggin could bias their descendents towards a neuronal fate, as BMP signaling induces gliogenesis. In this regard, work in the embryonic spinal cord and limb buds suggests that different concentrations of both SHH and BMPs are required for correct cell specification and proliferation (McMahon et al. (1998) Development 127:4395-4405; Liem et al., (2000) Development 127:4855-4866; Drossopoulou et al., (2000) Development 127:1337-1348). Thus, it is possible that Noggin is not inhibiting all BMP signaling but rather that it is a modulator required in cell fate decisions of dividing SVZ stem cells. More generally, the findings disclosed herein indicate that exposure of stem cells to Noggin and SHH, might induce them to produce large numbers of neurons. If so, this could be a productive source of new neurons to treat neurodegenerative diseases.

Conversely, the finding that the SHH-Gli pathway is active in adult neural stem cells raises the possibility that these cells are a source of adult tumors, as sporadic brain tumors has been proposed to derive from the inappropriate maintenance or activation of this pathway in the cerebellum (Goodrich et al., (1997) Science 277:1109-1113; Dahmane et al., (1999) Development 126:3089-3100; Wallace, (1999) Curr Biol 9:445-448; Weschler-Reya and Scott, (1999) Neuron 22:103-114) and elsewhere in the brain. Indeed, SHH signaling must cease to allow dividing cells (B/C cells) to differentiate (into A cells) and lack of cessation by any one of many possible mutations that activates the pathway (e.g., Xie et al. (1998) Nature 391:90-92; Reifenberger et al., (1998) Cancer Res 58:1798-1803); reviewed in Ruiz i Altaba, (1999) Development 126:3205-3216) may result in the initiation of tumorigenesis. Perhaps the preponderance of gliomas in adults results from an uncoupling of the simultaneous action of SHH and Noggin in the SVZ, which would result in an expansion of progenitors by an activated SHH-Gli pathway (through one of many possible mutations) that would then attempt to become glia in the absence of sufficient Noggin. These cells, however, could remain in a progenitor-like state given that the SHH-Gli pathway is constitutively activated, thus giving rise to a tumor. Nevertheless, why Gorlin's syndrome patients (with a single functional PTCH1 allele that can more easily have an activated SHH pathway due to loss of heterozygozity in single somatic cells) do not show a higher incidence of forebrain tumors, including gliomas, remains unclear.

The present demonstration of the role of Shh in the adult brain is consistent with prior findings in which the inappropriate activation of maintenance of the SHH-Gli pathway led to abnormal proliferation and CNS tumor formation (e.g.. Goodrich et al., (1997) Science 277:1109-1113; Dahmane and Ruiz i Altaba, (1999) Development 126:3089-3100; Weschler-Reya and Scott, (1999) Neuron 22:103-114; Rowitch et al. (1999) J Neurosci, 19:8954-8965); reviewed in Ruiz i Altaba, (1999) Development 126:3205-3216). A role of this pathway in the adult brain thereby provides a basis for the formation of adult tumors, especially since the adult SVZ has been proposed to be a possible site or origin of brain tumors (Lewis, (1968) Nature 217:974-975).

### EXAMPLES

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference to disclose and describe the methods and/or materials in connection with which the publications are cited.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Sonic Hedgehog Regulates SVZ Neurogenesis in the Post-Natal and Adult Brain

Animals, Dissections Cell-Sorting and Treatments. The SVZ of adult and post-natal mice were dissected as described previously (Lim et al.,(2000) Neuron 28:713-726, the contents of which are hereby incorporated by reference in their entireties). Cell culture was performed as described previously (Lim and Alvarez-Buylla, (1999) Proc. Nat. Acad. Sci USA 96:7526-7531), the contents of which are hereby incorporated by reference in their entireties. Purification of different SVZ cell types was performed by cell sorting using a FACSVantage (Becton Dickinson) with the following antibodies: B cells (GFAP, Dako), E cells (CD24, Pharmingen). Anti-SHH antibodies were purchased from the University of Iowa Hybridoma Bank. Recombinant octyl-modified SHH-N protein was obtained from Ontogeny Inc.

RNA, RT-PCR and In Situ Hybridization: RNA was isolated from whole SVZ dissections or from purified cells by as described previously (Lim et al.(2000) Neuron 28:713-726), the contents of which are hereby incorporated by reference in their entireties]. cDNA was synthesized and PCR performed for Gli1, Gli2, Gli3, Shh, Ptch1 and hprt as previously described (Dahmane and Ruiz i Altaba, (1999) Development 126:3089-3100, the contents of which are hereby incorporated by reference in their entireties). In situ hybridizations with anti-sense digoxygenin-labeled anti-Shh or anti-Gli1 probes were performed on fresh-frozen of perfused sections (Dahmane and Ruiz i Altaba, (1999) Development 126:3089-3100; Dahmane et al., (1997) Nature 389:876-881 the contents of which are hereby incorporated by reference in their entireties).

BrdU Incorporation and Immunohistochemical Analyses: Incorporation of BrdU and detection was performed as described previously (Lim et al.,(2000) Neuron 28:713-726; Lim and Alvarez-Buylla, (1999) Proc. Nat. Acad. Sci USA 96:7526-7531 the contents of which are hereby incorporated by reference in their entireties). Neuronal phenotype was determined by immunolabeling with TuJ1 antibodies (Babco) used at (1:1000). Nuclei were counterstained with Hoechst 33258 (Molecular Probes).

Thymidine Incorporation. 300,000 P3 SVZ cells were plated into uncoated wells into a 96-well plate in DMEM/F12/N2/B27/Gln/15 mM Hepes, pH 7.4 (Gibco) in the presence or absence of 5E1 or IgG (R&D systems) antibody at 4-5 ug/ml and cultured for a total of 44 hours. At this cell density, aggregates of SVZ cells form. At 27 hours, 2 uCi of [³H]-Thymidine was added to each well. Cells were collected onto glass filters with a Tomtec 96 cell harvester, and [³H]-Thymidine incorporation measured with a betaplate filter counter.

### Example 2

### SHH-GLI Signaling Regulates the Behavior of Neural Stem Cells in Cooperation with EGF

Animals, mutants, dissection, explants and treatments. Swiss-Webster mice were used unless otherwise specified. The Shh (Chiang et al., (1996) Nature 383, 407-413), Gli1 (Park et al., (2000) Development 127, 1593-1605) and Gli2 (Mo et al., (1997) Development 124, 113-123.) mutants from our colony were in this background. Cortical explants were prepared as previously described (Dahmane et al., (2001) Development 128, 5201-5212) and treated for 48 h. Anti-SHH antibodies were used at 4 μg/ml (University of Iowa Hybridoma Bank). Octyl-modified SHH-N protein was a kind gift from Ontogeny/Curis Inc.

BrdU incorporation, histology, immunofluorescence and in situ hybridization BrdU treatment (20 mg/kg, IP injection), microtome sections (12 μm), cryostat (10-18 μm), immunofluoresce, in situ hybridization and H&E staining was done as described (Dahmane et al., (2001) Development 128, 5201-5212). Explants were processed after a 1 h BrdU pulse in culture. BrdU was added to SVZ or cortical nsp cultures at 3 μM 16 hours or 7 hours prior to culture fixation, respectively. The following antibodies were used: beta III tubulin TuJ1 antibodies (1/300; Babco), Nestin (1/100, Becton Dickinson), activated caspase 3 (1/50, R&D Systems), GFAP (1/500, Sigma), O4 (1/40; Roche). Clones 224 and 53 derive are being characterized and were used as markers of vz/svz cells. Sense probes confirmed the specificity of in situ hybridizations. All P values were obtained with the Student's t-test.

Cell sorting The SVZ of adult and post-natal mice were dissected, cells cultured and sorted on a FACSVantage (Becton-Dickinson) as described (Doetsch et al., (1999) Neuron 36, 1021-1034); (Lim and Alvarez-Buylla, (1999) Proc Natl Acad Sci USA 96, 7526-31); (Lim et al., (2000) Neuron 28, 713-726). Antibodies used were biotinylated mCD24 antibody for E cells (1/10; Pharmingen), rabbit GFAP antibody for B cells (1/100; DAKO). Cells were then labeled with streptavidin-Cy2 at and anti-rabbit F(ab)2 at (Jackson Immunoresearch). Cells were washed 3 times with PBS and resuspended in PBS at 500,000 cells/ml. In the postnatal SVZ, the purification of B cells is contaminated with a number of C cells.

RT-PCR and genotyping Conditions and sequences were as described (Dahmane et al., (2001) Development 128, 5201-5212). Other primers and conditions used were: mGli2-1 (forward): gca gct ggt gca tca ta; mGli2-2 (reverse): cgg tgc tca tgt gtt tg with Tm=55° C. for 35 cycles, producing an 828 bp band for the wild type allele and a 913 bp band for the Gli2 mutant allele. Primers for Ihh and Dhh were used at Tm=58° C. and gave expected band sizes of 267 bp for Ihh and 311 bp for Dhh. Ihh1: ggc cat ctc tgt cat gaa cc; Ihh2: cag cca cct gtc ttg gca gc; Dhh1: gtg cgc aag caa ctt gtg cc; Dhh2: gaa tcc tgt gcg tgg tgg cc. For SVZ dissections or for purified SVZ cells (Lim et al., 2000), 5,000-10,000 selected cells were used with the SMART III protocol (Clontech).

Neurospheres (nsps) Cortical and SVZ nsps were obtained by standard procedures (Doetsch et al., (1999) Cell 97, 703-716). The cells were incubated in neurosphere medium (Neurobasal Medium (GIBCO) supplemented with N2 (GIBCO), 2 mM glutamine, 0.6% (w/v) glucose, 0.02 mg/ml insulin, antibiotics and 15 mM HEPES) with 10 ng/ ml of EGF (human recombinant, GIBCO) and 10 ng/ml of bFGF (Upstate Biotech) unless otherwise noted. For proliferation assays, nsps were plated at 3000 cells/well onto polyornithine/laminin coated Lab-Tek chamber slides (Nunc) in the presence of EGF and FGF and grown for 1 week. For differentiation, growing nsps were plated at 20,000 cells/well onto polyornithine/laminin coated (10 mg/ml) Lab-Tek chamber slides without growth factors 5-8 days. For cloning assays cells were plated by dilution at 1cell/well in 96 well plates (Nunclon) with 50% conditioned media: 50% nsp defined media containing EGF (10 ng/ml) and bFGF (10 ng/ml). The number of and size of cloned nsps was counted after one week in culture. SVZ nsps were made from the lateral walls of the lateral ventricle of postnatal or adult mice. SVZ nsps were grown medium containing 10 ng/ ml EGF.

Thymidine incorporation 300,000 P3 SVZ cells were plated into uncoated wells into a 96-well plate in DMEM/F12/N2/B27/Gln/15 mM Hepes, pH 7.4 (Gibco) in the presence or absence of 5E1 or IgG (R&D systems) antibody at 4-5 μg/ml and cultured for a total of 44 h, forming aggregates. At 27 h, 2 μCi of ³H-Thymidine was added to each well. Cells were collected onto glass filters with a Tomtec 96 cell harvester, and ³H-Thymidine incorporation measured with a betaplate filter counter.

In vivo cyclopamine treatment Cyclopamine (Toronto Research Biochemicals) was used at 1 mg/ml conjugated with 2-Hydropropyl-β-Cyclodextrin (HBC (Sigma); prepared as a 45% solution in PBS). Five to ten week-old inbred C57B16/j mice were injected intraperitoneally for one week with HBC alone as control or cyc at 10 mg/kg/day. The day following the last injection, the mice were pulsed for 2 h with BrdU (20 mg/kg, IP injection). Immunofluorescence of cryostat sections was as described (Dahmane et al., (2001 Development 128, 5201-5212). The stainings were digitally recorded using a cooled CCD camera-equiped Axiophot (Zeiss) and the BrdU⁺/DAPI⁺ nuclei counted within the lateral wall of the lateral ventricles. For the in vivo treatment followed by the preparation of nsps, pregnant mothers (E12.5) or P4 pups were injected for 5 days with HBC alone or cyc at 10 mg/kg/day and cortical nsp from E17.5 embryos or SVZ nsp from P9 animals were made, respectively.

Results

Gli2 Mutant Mice Have Reduced Dorsal Brain Structures and Germinative zones

Gli1 and Gli2 can mediate positive actions of SHH signaling, whereas Gli3 has mostly an antagonistic relationship, although Gli3 can also have positive functions (Brewster et al., (1998) Nature 393, 579-583); Dai et al., (1999) J Biol Chem 274, 8143-8152); Sasaki et al., (1999) Development 126, 3915-3924). In mice, Gli1 appears to be functionally redundant with other Gli proteins (Park et al., (2000) Development 127, 1593-1605; Bai and Joyner, (2001) Development 128, 5161-5172); Bai et al., (2002) Development 129, 4753-4761), and in some contexts Gli2 can functionally overlap with Gli3 (Mo et al., (1997) Development 124, 113-123). While loss of Gli3 rescues most of the Shh mutant phenotype (e.g. Litingtung and Chiang, (2000) Nat Neurosci 3, 979-985), and here it appears that it is the repressor form of Gli3 at play (Persson et al., (2002) Genes Dev. 16, 2865-78), there is also an involvement of SHH mediated positively by Gli2 in ventral patterning (Ding et al., (1998) Development 125, 2533-2543; Matise et al., (1998) Development 125, 2759-2770). Here we have focused on the role of Gli2 in the dorsal brain.

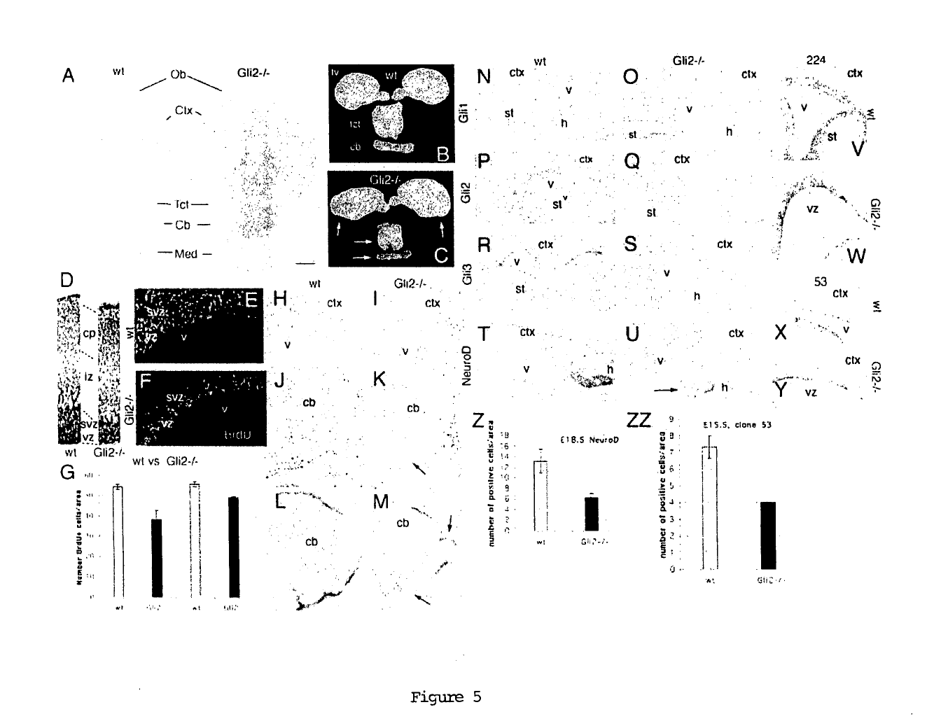

Gli2 null homozygotes die at birth displaying shortened bodies and faces as well as defects in the skeleton, viscera and ventral neural tube (Mo et al., (1997) Development 124, 113-123; Ding et al., (1998) Development 125, 2533-2543; Matise et al., (1998) Development 125, 2759-2770). We have analyzed their brains at embryonic day (E) ˜15.5 and ˜18.5, when the basic morphology and cytoarchitecture of the adult brain begin to be visible. Gli2−/31 mice display a brain phenotype with expanded but thinner telencephalic vesicles, most clearly seen posteriorly and overtly reduced tectum and cerebellum, as compared with normal wild type littermates (FIG. 5A-C).

Histological analyses of H&E stained sections of 4 animals showed that E18.5 Gli2−/− telencephalic vesicles have a thinner proliferative zone (˜30-50% reduction of the vz/svz) in Gli2−/− versus wild type littermate cortices; FIG. 1D) and an apparently normal choroid plexus (not shown). Cell density and size appeared normal. The ballooning of the telencephalic vesicle could be due to the inability of tissue thinner than normal to sustain the same degree of intravesicular pressure. The intermediate zone and cortical plate layers appear of normal size and cell density. Analyses of BrdU incorporation showed that Gli2−/− mice have less precursor proliferation in the developing cortex as compared to wild type littermates (FIG. 5E-I), a result also observed at E15.5 (not shown), suggesting defects in neuronal as well as glial cell populations born at these various stages (Levers et al., (2001) J. Neurobiol 48, 265-277). The observed decrease is most notable in the deeper proliferative area (FIG. 5G), considered here for quantification purposes as the ‘cortical svz’ with a domain between 5 and 10 cell diameters from the ventricle. Within the anteroposterior extent of the Gli2−/− neocortex, however, we observed local variations without a clear pattern in the density of BrdU⁺ nuclei (FIG. 5I), suggesting an additional degree of disorder in these mutant mice. A reduction of the cerebellum was also evident on BrdU-labeled sections (FIG. 5 J, K), which showed fewer labeled cells, especially in the posterior region. TUNEL and activated Caspase-3 analyses did not show a difference in apoptotic cell death between Gli2−/−0 and wild type brains (not shown).

Gli2 Mutant Cortices Show the Loss of Gli1 and a Reduction in Gli3 and NeuroD Expression.

Absence of Gli2 function in the Gli2−/31 mice was found to lead to the complete absence of Gli1 transcripts in the developing cortex (FIG. 5N, O). A domain of Gli1 expression in the striatum, however, was still present in mutant brains (FIG. 5O). Expression of Gli3 was also diminished as compared to wild type littermates (FIG. 5R, S). Expression of wild type and mutant Gli2 transcripts was not altered, with the exception that their expression in the vz/svz comprised fewer cells as these regions are smaller (FIG. 5P, Q). To quantify this phenotype we performed in situ hybridization with NeuroD, which marks neuroblasts, and with two clones, 224 and 53, obtained from an unrelated cortical screen and that strongly and homogeneously label the ventricular areas. The expression of NeuroD, 224 and 53 (FIG. 5T-Y) showed a reduction of neuroblasts and vz/svz cells, respectively, by ˜50% in Gli2−/− cortex versus that of wild type littermates (FIG. 5Z, ZZ). A reduction was also found in ventral but not in medial areas of the telencephalic vesicle (not shown). A similar decrease in NeuroD expression was detected in the cerebellum, which showed abnormal early foliation and more pronounced posterior defects (FIG. 5L, M). Neocortical pattern, however, appeared largely unchanged as the expression of the Pax6 anteroposterior gradient and of Dlx2 ventrally were not grossly affected, with the exception that fewer cells in the vz/svz expressed these genes (not shown). The hippocampus was also smaller in Gli2−/− versus wild type littermates (FIG. 5T, U). Together, these findings show that Gli2, normally expressed in the vz/svz, is required to control the production of the number of dorsal brain precursors and subsequently of neuroblasts.

Gli2 Mutant Explants Retain Responsiveness to SHH

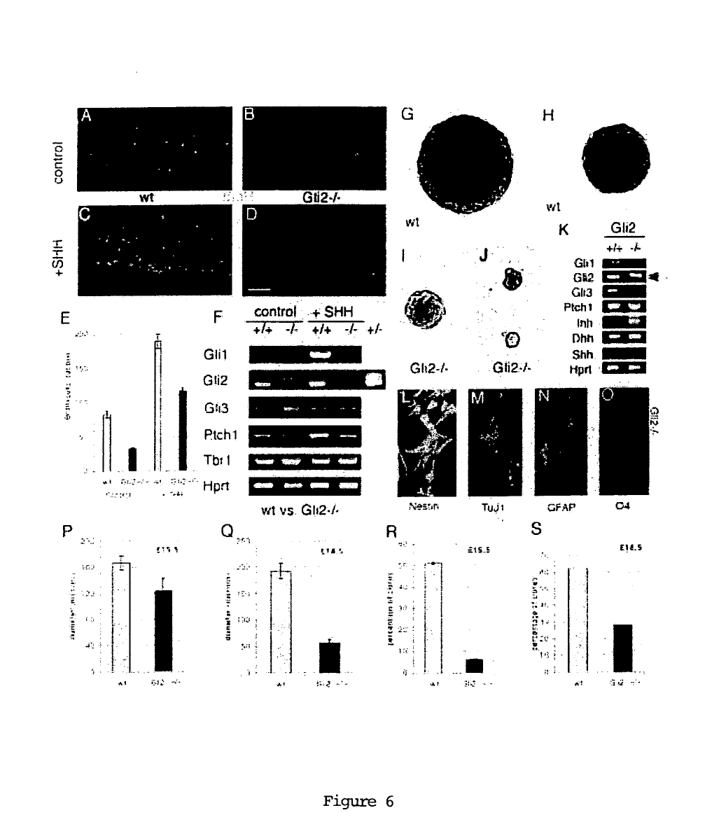

Analyses of tissue explants from the parietal neocortical region grown in vitro in serum-free media (Dahmane et al., (2001) Development 128, 5201-5212) showed that Gli2−/− explants proliferate ˜50% less than wild type ones (FIG. 6A,B,E), consistent with the smaller vz/svz of Gli2−/− brains. However, both wild type and Gli2−/− explants were able to respond to exogenous SHH treatment by increasing BrdU incorporation (FIG. 6C,D,E). RT-PCR analyses of gene expression in control and treated explants (FIG. 6F) showed that mutant cells lack significant expression of Gli1. After treatment with SHH, however, there is both an increase in Gli1 expression and a slight increase in Ptchl transcripts. These results show that Gli2 is not necessary for a response to SHH and that in the absence of Gli2, Gli1 can be slightly upregulated possibly accounting for the proliferative response of the explants to SHH treatment, as one copy of Gli1 knocked-in into the Gli2 locus can rescue the Gli2 null phenotype (Bai et al., (2002) Development 129, 4753-4761.

Gli2 Mutant Brains Produce Few and Small Neurospheres

The results with Gli2 null embryos and explants show that Gli2 is required for precursor behavior, but does not address whether Gli2 affects the behavior of stem cells, as these may represent a minority population (Tropepe et al., (1999) Dev Biol. 208, 166-88); see Temple, (2001) Nature 414, 112-7). To test a possible role of Gli2 in cortical stem cell behavior, and lacking specific markers of neural stem cells, we have cultured cortical neurospheres (nsps) from wild type and Gli2−/− brains. Each nsp is derived from an individual founder stem cell—which can self-renew and produce progeny of different types—and their presence and number are thus good indications of the existence and abundance of neural stem cells. Nsps are thought to contain less than 10% of stem cells while the bulk is made up by committed progenitors as well as by an undetermined number of differentiated cells (e.g. Tropepe et al., (1999) Dev. Biol 208, 166-88). We were able to obtain nsps from wild type and Gli2−/− cortices (FIG. 6G-J) and in both cases these were Nestin⁺ (FIG. 6L) and could be passaged several times. This, together with the fact that they could be differentiated into TuJ1⁺ neurons, O4⁺ oligodendrocytes and GFAP⁺ astrocytes (FIG. 6M-O) confirmed the presence of stem cells. Mutant nsps of first, second or later passages were smaller and blebbier than wild type ones (FIG. 6I, J, P, Q). Gli2−/− nsp cells were also found to be more delicate during the manual dissociation process. After the sixth passage, Gli2−/− nsps were rare and died soon, indicating that stem cells lacking Gli2 show compromised viability, which was more evident in those from E18.5 than in those from E15.5 (FIG. 6P, Q). Cloning assays testing for stem cell self-renewal showed that at frequencies of 1 cell per well, there was a 2-fold decrease in the number of stem cells able to form nsps, as compared to that of wild type littermates, at E18.5 (FIG. 6S) and a 10-fold difference at E15.5.

Analyses of gene expression in wild type and Gli2−/− nsps confirmed the absence of Gli1 expression in Gli2−/− cortical cells (FIG. 6K). Mutant cells also showed the upregulation of the expression of Indian hedgehog (Ihh) and Desert hedgehog (Dhh), the other two mammalian Hh genes. This is unexpected as Ihh and Dhh have not been reported to be expressed in the mouse brain, although they are also detected by RT-PCR in explants and in fresh cortical tissue (not shown). Shh was expressed in both wild type and mutant nsps at low levels (FIG. 6K), while Gli3 expression was reduced (FIG. 6K). Together, these results with nsps and explants indicate that Gli2 is required for normal precursor proliferation as well as for the viability of nsp-forming neural stem cells at mid and late gestation periods.

Shh Mutant Cortices Yield Few and Small Neurospheres

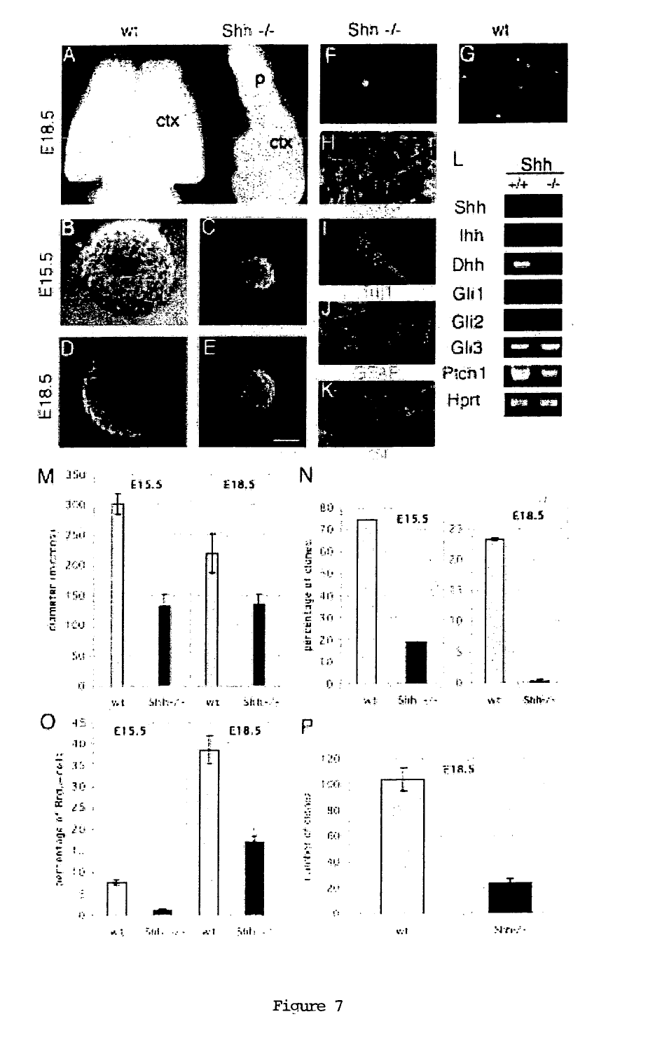

To investigate if Gli2 could be acting in the SHH pathway in cortical cells, as it is in the early embryo (Bai et al., (2002) Development 129, 4753-4761), we have analyzed precursor and stem cell behavior in the cortex of Shh−/− mice (Chiang et al., (1996) Nature 383, 407-413) to compare the results to those obtained with Gli2−/− animals. Shh−/− mice show severe holoprosencephaly (Chiang et al., (1996) Nature 383, 407-413) and the brain lacks ventral structures, consisting of reduced dorsal regions that includes a forebrain cortex that can be identified and isolated (FIG. 7A), and which expresses normal neocortical markers such as Emx1 and Thr1 (Chiang et al., (1996) Nature 383, 407-413; Dahmane et al., (2001) Development 128, 5201-5212 and not shown). Stem cells are present in the cortex of E15.5 and E18.5 Shh−/− brains as assessed by the formation of nsps (FIG. 7C,E). As in the case of Gli2 mutants, these were smaller than those from wild type cortices, containing also fewer BrdU⁺ cells (FIG. 7B-E,F,G,M,O). Analyses of gene expression confirmed the loss of Shh transcripts in the Shh−/− nsp and a slight decrease in Ptch1 and Dhh expression, whereas the expression of Ihh, Gli1 and Gli2 were unchanged (FIG. 7L). Gli3 expression was slightly higher in the absence of Shh (FIG. 3L). Shh−/− nsps, like the wild type ones, could be differentiated into TuJ1⁺ neurons, O4⁺ oligodendrocytes and GFAP⁺ astrocytes at E15.5 and E18.5 (FIG. 7H-K and not shown). Cloning assays showed that Shh−/− nsps contain ˜¼-⅕^{th} the number of nsp-forming stem cells as compared to wild type nsps, at E15.5 and E18.5 (FIG. 7N). Thus, SHH, like Gli2, is required for neural stem cell maintenance but it is also likely involved in the survival of the more abundant precursors.

In Vivo Reduction of HH Signaling with Cyclopamine Increases the Number of Cortical Stem Cells

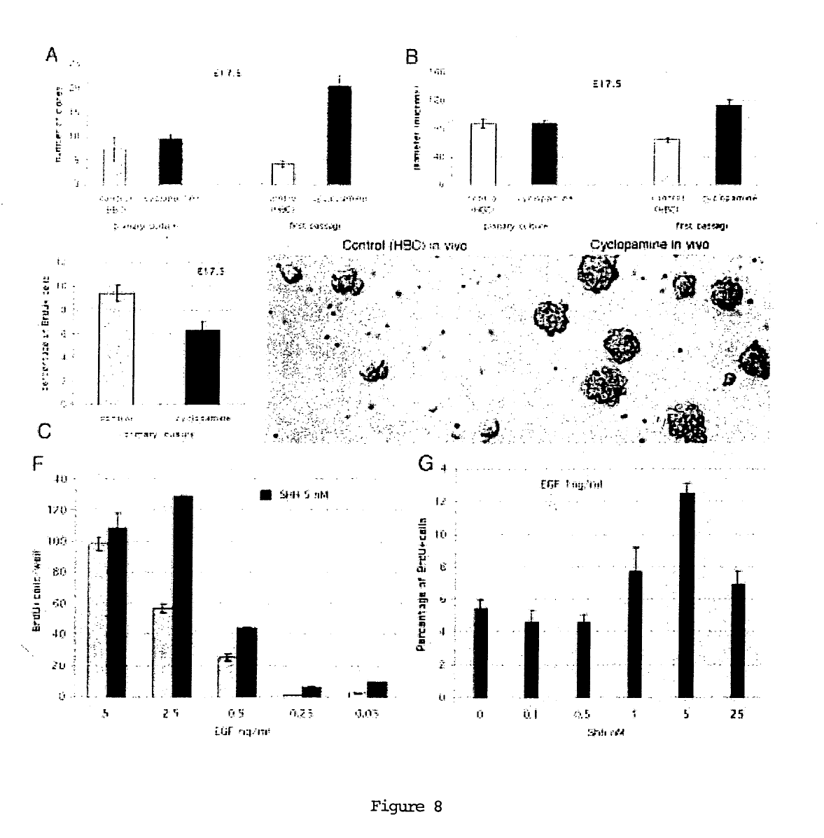

To test the role of HH signaling on neural stem cells from the neocortex in vivo we have treated pregnant mice with cyclopamine (cyc), a selective HH signaling inhibitor (Incardona, et al., (1998) Development 125, 3553-3562; Cooper et al., (1998) Science 280, 1603-1607; Taipale et al., (2000) Nature 406, 1005-1009). Intraperitoneal injections of cyclodextrin-coupled cyc has been previously shown to affect HH signaling in the gastric mucosa (van den Brink et al., (2001) Gastroenterology 121, 317-28). Daily injection of 10 mg/kg/day of cyc for 5 days, starting at E12.5, resulted in E17.5 embryos that were morphologically normal, having passed by E12.5 the period when inhibition of SHH signaling results in cyclopia (Chiang et al., (1996) Nature 383, 407-413). The neocortices of cyc and carrier-only treated, control embryos were dissected and nsp cultures prepared in normal nsp media—in the absence of cyc—to measure the number of stem cells present. Analyses of the number of nsps in primary and secondary cultures showed that there was a clear increase in the number of nsps in cyc vs control cultures (FIG. 8A,D,E), with the greatest increase (˜4-fold) seen after the first passage. Measurement of the size of the resulting nsps showed that the cyc-treated ones were slightly larger after a first passage (FIG. 8B,D,E). However, consistent with the decrease in BrdU incorporation seen in Shh null nsps, primary cultures of cyc-treated nsps showed less proliferation than control primary nsp cultures only when cultured with residual amounts of EGF present in the conditioned media used (FIG. 8C). Cyc treatment had no effect on the proliferative behavior of nsps grown with high levels of EGF (10 ng/ml; not shown). These results indicate that reduction of HH signaling in vivo (and a consequent reduction in Gli1 expression (not shown)) results in a decrease in proliferation accompanied by an increase in the number of nsp-forming neural stem cells that have a slightly larger in vitro proliferative potential. Moreover, they suggest that SHH signaling may not be required for proliferation under saturating levels of EGF.

SHH and EGF Synergize

The decrease in precursor proliferation in cyc-treated primary cultures or Shh−/− nsps raised the possibility that SHH could cooperate with the growth factors required for nsp formation and expansion. EGF can sustain nsp growth at late embryonic stages while at earlier stages FGF is required (Tropepe et al., (1999) Dev Biol. 208, 166-88; Martens et al., (2000) J. Neurosci. 20, 1085-1095). Addition of SHH (5 nM) to standard nsp media containing 10 ng/ml of EGF had no effect on E18.5 nsp proliferation, and nsp growth was not sustained by media supplemented with SHH (at 5nM) without FGF or EGF (not shown). E18.5 wild type cortical nsps were then selected in media containing 10 ng/ml EGF without FGF and passed and grown at different concentrations of EGF. Interestingly, SHH (5 nM) synergized with EGF at concentrations between 2.5 and 0.05 ng/ml, which were still able to promote nsp growth (FIG. 8F). A synergism was not detected at 5 ng/ml of EGF, suggesting that at this high level it can bypass the effects of SHH or that it saturates the response. Conversely, growth of nsps at 1 ng/ml of EGF showed a concentration-dependent increase in proliferation by SHH between 1 and 5 nM (FIG. 8G). To further test this synergism we attempted to make nsps from Shh null E18.5 cortices in the presence of EGF (10 ng/ml) without FGF. Under these conditions, we were unable to obtain significant primary nsp formation from these animals, whereas control wild type, littermate nsps grew normally (not shown). These results suggest a required synergism of SHH and EGF for nsp formation and proliferation by neural stem cells.

The Shh and Gli Genes are Expressed in the Postnatal and Adult SVZ

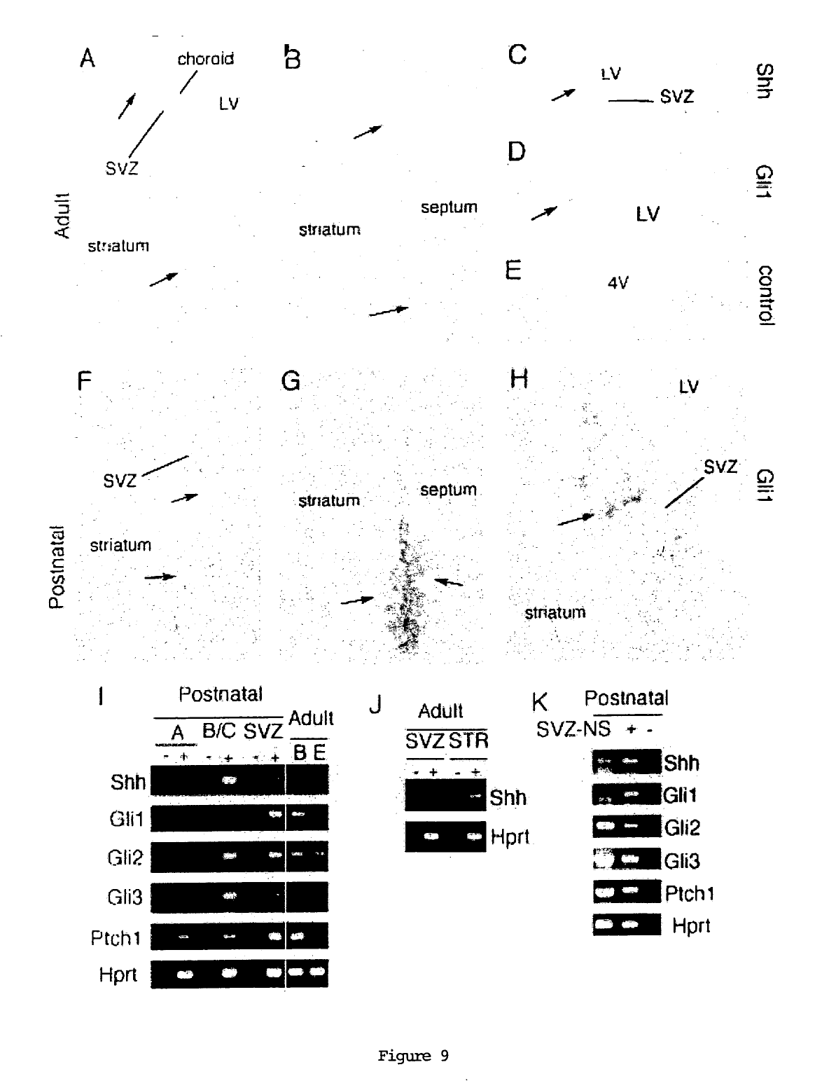

To test if SHH signaling could be a common factor affecting the behavior of different neural stem cell populations we analyzed the well-characterized neural stem cells in the postnatal and adult SVZ of the lateral ventricle. We tested for the expression of the Shh and Gli1 genes, the latter being a loyal responder of SHH signaling (reviewed in Ruiz i Altaba et al.,2002a), in the SVZ. Both Shh and Gli1 were detected at low levels in the lateral wall of the SVZ by in situ hybridization (FIG. 9A-H and not shown). Expression of these genes in the adult striatum and septum, was detected at much lower levels (FIG. 9A,B). Within the SVZ, the labeling was mostly confined to the lateral wall of the lateral ventricle, where neurogenesis occurs. However, Gli1 expression suggests that the SVZ may be regionalized as it was more highly expressed in the ventral region. Interestingly, Gli1 was also expressed in the ventral medial wall, possibly defining a new germinative zone (FIG. 9A,B,F,G). Most brain cells did not express Shh, indicating the specificity of the hybridizations (FIG. 9E and not shown). Within the SVZ, we found expression of Shh throughout most of its thickness (FIG. 9C), while that of Gli1 appeared to be at higher levels in the deep SVZ (FIG. 9D, H).

Gene Expression in Sorted SVZ Cells

To better define the cells that express Shh and those that respond to it, dissociated and sorted post-natal (Lim and Alvarez-Buylla, (1999) Proc Natl Acad Sci USA 96, 7526-31) and adult (Lim et al., (2000) Neuron 28, 713-726) SVZ cells were used to perform RT-PCR assays testing for the expression of genes involved in SHH signaling. Like Gli1, the transcription of the gene encoding the SHH receptor Ptchl is SHH-responsive (Goodrich et al., (1996) Genes Dev. 10, 301-312). In contrast, Gli2 activation by SHH is context-dependent, whereas Shh/Gli1 and Gli3 often have an antagonistic relationship (e.g. Ruiz i Altaba, (1998) Nature 393, 579-583); Litingtung and Chiang, (2000) Nat Neurosci 3, 979-985). In the postnatal SVZ, sorted neuroblasts (A cells) only expressed low level of Gli2 and Ptch1 but not Shh or Gli1, whereas the fraction containing astrocytes and transiently amplifying (B and C) cells expressed high levels of Shh, Gli2, Gli3 and Ptch1, but also Gli1, albeit at low levels (FIG. 9I). In the adult, ependymal (E) cells expressed only low levels of Gli2 and Ptch1, whereas SVZ astrocytes (B cells) expressed high levels of Gli1, Gli2 and Ptch1. We did not detect expression of Shh or Gli3 in sorted adult astrocytes or ependymal cells (FIG. 9I).

Since SHH acts through Gli1/2, and both Gli1 and Ptch1 are responsive to SHH (e.g. reviewed in Ruiz i Altaba et al., (2002a) Nat Rev Neurosci 3, 24-33), our data suggests that post-natal cells early in the lineage (SVZ astrocytes and transiently amplifying precursors—B and C cells—) express the signaling molecule and respond to it. SHH could, in principle, also affect neuroblasts and ependymal cells, as they express Gli2 and Ptch1. In adults, targets of SHH signaling are SVZ astrocytes (B cells), as these express Gli1 and Ptch1, although we cannot rule out the possibility that transiently amplifying precursors (C cells) also respond. The expression of Shh in the adult SVZ (FIG. 9J), but its absence from isolated B or E cells suggests that either other SVZ cells express it, possibly amplifying precursors (C cells), or that rare messages were lost during cell sorting and cDNA amplification. To confirm and extend these findings, we have analyzed gene expression in postnatal P7 SVZ nsps grown in standard media containing EGF. SVZ nsp cells express Shh, Gli1 and Ptch1 (FIG. 9K), further indicating that SHH is an endogenous factor. Together, gene expression analyses with sorted SVZ cells and nsps suggest that SVZ astrocytes and possibly transiently amplifying precursors respond to SHH.

SHH Increases Proliferation and Neurogenesis From SVZ Cultures

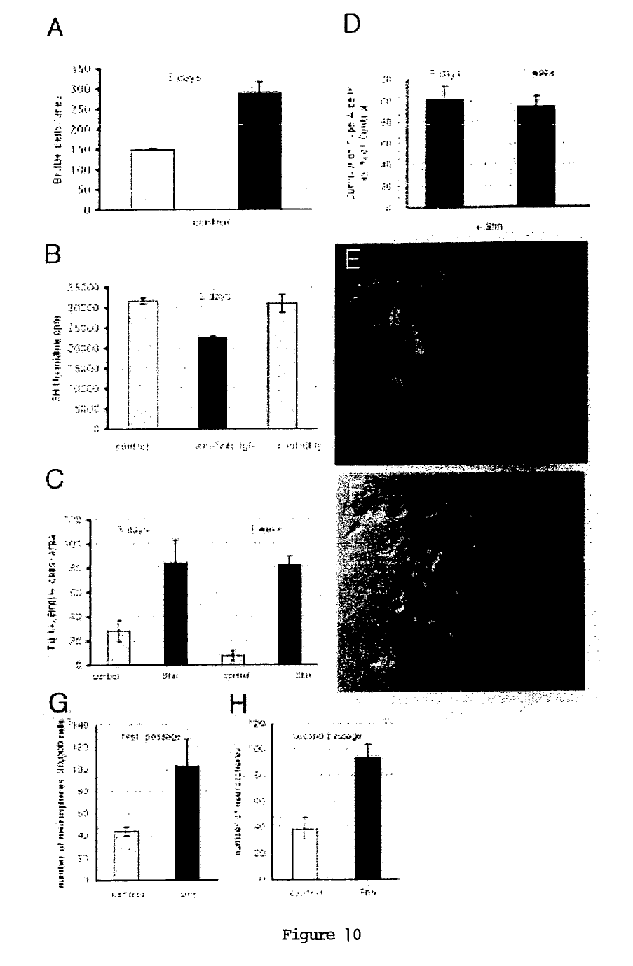

To test the role of SHH on SVZ cells, we plated dissociated post-natal P5 SVZ cells on a quiescent astrocytic monolayer as previously described (Lim et al., (2000) Neuron 28, 713-726; Lim and Alvarez-Buylla, )1999) Proc Natl Acad Sci USA 96, 7526-31) in the absence of exogenous FGF or EGF. Addition of SHH at the start of the culture period doubled the number of BrdU⁺ cells measured after five days (FIG. 10A). The requirement of SHH for SVZ cell proliferation in vitro was tested by making aggregates of dissociated post-natal SVZ cells in the absence of the astrocytic monolayer, and treating them with anti-SHH monoclonal antibody that we have previously used (Dahmane and Ruiz i Altaba, (1999) Development 126, 3089-3100), in the presence of ³H-thymidine. Addition of anti-SHH antibody decreased proliferation by ˜30% after two days as compared to sibling cultures treated with an isotype-matched unrelated antibody (FIG. 10B). Dissociated adult SVZ cells plated onto astrocyte monolayers in a defined, serum-free medium proliferate to form colonies of Tuj1⁺ neuroblasts. In this assay, addition of SHH increased the number of newly born Tuj1⁺ neurons by ˜3-fold after 3 days and by ˜10-fold after 1 week (FIG. 10C,E,F). The increase could be compounded by a survival effect on stem cells as the number of new neurons in the control sample decreases between 3 and 7 days. This effect does not appear to be a direct action of SHH on neuroblasts as SHH treatment of such purified cells did not increase their numbers in culture as compared to controls, suggesting that SHH affects neither their survival nor their proliferation (FIG. 10D). On the contrary, these findings suggest that SHH acts on amplifying precursors (C cells) to increase their proliferation and thus to increase the rate of neurogenesis.

SHH Increases the Number of Neurosphere-Forming Cells in SVZ Cultures

To measure effects of SHH on SVZ stem cells in vitro we have treated primary SVZ cultures on astrocytic monolayers with recombinant SHH and assayed for the number of resulting nsp-forming cells in the absence of SHH. After 4 days of SHH treatment, an equal number of treated and control untreated SVZ cells were seeded for nsp cultures with EGF but without SHH. After 1 week of growth, cultures from SHH-treated SVZ cells had 2-fold the number of nsps (FIG. 10G). This difference was maintained after the further passage of the nsps, and these were of similar size to the controls ones (FIG. 10H and not shown). Because the bulk of nsp-forming cells in the presence of EGF is made up by C cells (Doetsch et al., (2002) Neuron 36, 1021-1034), we interpret these data to suggest that SHH induces amplifying precursor proliferation, which under the influence of EGF, can self-renew and give rise to distinct differentiated cell types, thus behaving as stem cells. We cannot rule out the possibility that SHH is also acting to enhance proliferation of the SVZ astrocytes, which behave as the rare stem cells in vivo (Doetsch et al., (1999) Cell 97, 703-716).

In Vivo Reduction of HH Signaling with Cyclopamine Increases the Number of Neurosphere-Forming SVZ Stem Cells

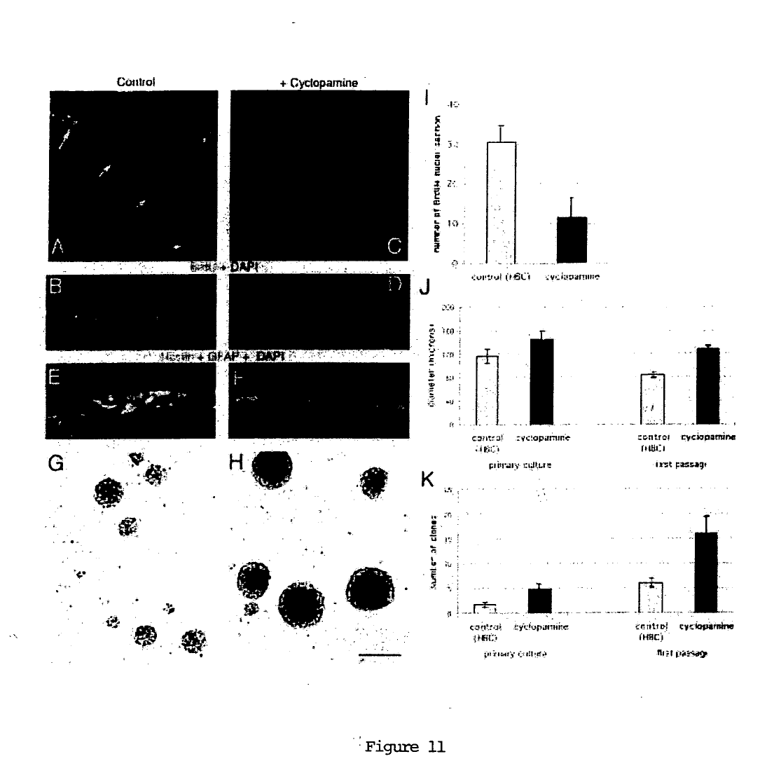

To test the role of SHH in the regulation of SVZ stem cells in vivo, we treated 2month-old adult mice with cyc (10 m/kg/day) for 7 days. The mice appeared normal throughout the treatment and were given a 2 h BrdU pulse before sacrifice. Although there was a variability in the response of individual mice to cyc, analyses of BrdU⁺ cells in the brain showed that in the SVZ, cyc-treatment caused a marked decrease (˜3-fold) in the number of dividing cells (FIG. 11A-D,I), suggesting that inhibition of SHH impairs the proliferation of abundant cell types, possibly amplifying precursors. The SVZ of the treated mice appeared morphologically normal and its cells expressed GFAP and Nestin in normal patterns (FIG. 11E, F). However, we detected a ˜2-fold increase of Nestin expression in the adult brains treated with cyc that showed little BrdU staining when compared with control brains.

To test how in vivo cyc treatment may affect the number of SVZ cells with stem cell potential, we made nsp cultures from the SVZ of cyc-treated and control P9 mice. We chose this age because there are proportionally more nsp-forming cells at this time than in the adult. We found that in vivo cyc-treatment decreased proliferation as in adults (not shown and see above) and increased the number of nsp-forming cells (subsequently cultured without cyc). This increase was more evident (˜3-fold) after the first passage (FIG. 11G,H,K). Nsps derived from cyc-treated animals were 20-30% larger (FIG. 11G,H,J). Cyc-treatment in vivo thus amplifies the SVZ nsp-forming cell pool. Nsp-forming cells may be derived from both SVZ astrocytes and transiently amplifying precursors, which after exposure to EGF can adopt stem cell properties (Doetsch et al., 2002). The overall decrease in BrdU incorporation following cyc treatment (FIG. 11I) suggests that following reduction of HH signaling, amplifying precursors proliferate less and remain in the SVZ. The number of these cells and of resident SVZ astrcytes, together with those derived from divisions of the latter, (which are Gli1+ and could be affected by SHH), may then be larger than the steady-state number of nsp-forming (B and C) cells normally present in the SVZ. This would then account for the increase in in vitro nsps after prolonged in vivo cyc treatment that we observe.

The results shown above demonstrate that SHH acts on cells with stem cell potential both from the developing cortex at mid and late gestation periods and of the postnatal and adult SVZ. In addition, the data also shows that SHH synergizes with EGF. Furthermore, the data suggests that SHH may act on nsp-forming cells. These may be SVZ astrocytes (B cells), which express Gli1 and Ptch1, as well as C cells, which form the bulk of nsps made in vitro in the presence of EGF. The ability of SHH signaling to increase the production of neurogenic precursors from stem cells raises the possibility of its use for the expansion of distinct cell populations as a strategy for treating neurodegenerative diseases. This strategy could also apply to treating mental deficits associated with viable mutations affecting SHH signaling.

What is claimed is:

1. A method of proliferating a mammalian cell from the Central Nervous System (CNS) and its subsequent differentiation to become a neuron comprising culturing the cell in the presence of an agent that stimulates the SHH-GLI pathway.

2. The method of claim 1, wherein the agent is a hedgehog or an active fragment thereof.

3. The method of claim 2, wherein the hedgehog is selected from the group consisting of sonic hedgehog, desert hedgehog and Indian hedgehog.

4. The method of claim 2, wherein the agent is combined with a therapeutically effective amount of a growth factor.

5. The method of claim 4, wherein the growth factor is Epidermal Growth Factor (EGF).

6. The method of claim 1, wherein the mammalian cell is a brain stem cell.

7. The method of claim 6, wherein the brain cell is either an adult, perinatal or post-natal neural stem cell.

8. The method of claim 7, wherein the adult, perinatal or post-natal neural stem cell is a human adult, perinatal or post-natal neural stem cell.

9. The method of claim 6, wherein the brain cell is a mouse subventricular stem cell.

10. A method of generating a neuron from an adult, perinatal or post-natal neural stem cell comprising culturing the stem cell in the presence of an agent that stimulates the SHH-GLI pathway.

11. The method of claim 10, wherein the agent is a hedgehog or an active fragment thereof.

12. The method of claim 11, wherein the hedgehog is selected from the group consisting of sonic hedgehog, desert hedgehog and Indian hedgehog.

13. The method of claim 11, wherein the agent is combined with a therapeutically effective amount of a growth factor.

14. The method of claim 13, wherein the growth factor is Epidermal Growth Factor (EGF).

15. A method for treating and/or preventing a neurologic or neurodegenerative disease, disorder or condition in a mammal, comprising transplanting into the brain of the mammal a neuronal cell prepared by the method of claim 1.

16. The method of claim 15, wherein the mammal is a human.

17. The method of claim 15, wherein the neurologic condition is due to an injury.

18. The method of claim 17, wherein the injury is a spinal cord injury.

19. The method of claim 15, wherein the neurologic condition is due to brain damage arising from trauma to the head or stroke.

20. The method of claim 15, wherein the neurodegenerative disease is selected from the group consisting of Alzheimer's disease, Huntington's disease, Parkinson's Disease, schizophrenia, multiple sclerosis, amyotropic lateral sclerosis (ALS), progressive supranuclear palsy, Creutzfeldt-Jakob Disease, epilepsy, and dementia.

21. A method for treating and/or preventing a neurologic or neurodegenerative disease, disorder or condition in a mammal, comprising transplanting into the brain of the mammal an expression vector that encodes sonic hedgehog or an active fragment thereof.

22. A method for enhancing the neuronal content of an adult mammalian brain comprising transplanting into the brain of the mammal a neuronal cell prepared by the method of claim 1.

23. A method of delivering or expressing a gene in the CNS or brain of a subject comprising transplantation into the brain of the subject a neuronal cell preparation prepared by the method of claim 1, wherein the neuronal cells have been genetically modified to encode a specific gene.

24. A method of treating tumors comprising administering an agent that blocks endogenous SHH and/or GLI signaling.


SEQUENCE LISTING (14 sequences)

SEQ ID NO: 1 - DNA, 1575 bp, Homo sapiens
gcgaggcagccagcgagggagagagcgagcgggcgagccggagcgaggaagggaaagcgcaagagagagcgcacacgcacacacccgccgcgcgcactcgcgcacggacccgcacggggacagctcggaagtcatcagttccatgggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaagccggggggccgggggaggggcgcgggagggggc

SEQ ID NO: 2 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 3 - DNA, 1314 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaagctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctga

SEQ ID NO: 4 - protein, 429 aa, Mus musculus
FLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 5 - DNA, 1714 bp, Rattus norvegicus
ttaaaatcaggctctttttgtcttttaattgccgtctcgagacccaactccgatgtgttccgttaccagcgaccggcagcctgccatcgcagcccctgtctgggtggggatcggagacaagtcccctgcagcaacagcaggcaaggttatataggaagagaaagagccaggcagcgccagagggaacgaacgagccgagcgaggaagggagagccgagcgcaaggaggagcgcacacgcacacacccgcgcgtaccagctcgcgcacagaccggcgcggggacggctcgcaagtcctcaggttccgcggacgagatgctgctgctgctggccagatgttttctggtggcccttgcttcctcgctgctggtgtgccccggactggcctgtgggcccggcagggggtttggaaagaggcagcaccccaaaaagctgacccctttagcctacaagcagtttatccccaacgtagccgagaagaccctaggggccagcggccgatatgaagggaagatcacaagaaactccgaacgatttaaggaactcacccccaattacaaccccgacatcatatttaaggatgaggaaaacactggagcagaccggctgatgactcagaggtgcaaagacaagttaaatgccttggccatctccgtgatgaaccagtggcctggagtgaagcttcgagtgactgagggctgggatgaggacggccatcattcagaggagtctctacactatgagggtcgagcagtggacatcaccacgtctgacagggaccgcagcaagtatggcatgctggctcgcctggctgtggaggctggattcgactgggtctactatgaatccaaagctcgcatccactgctctgtgaaagcagagaactccgtggcggccaaatctgacggctgcttcccgggatcagccacagtgcacctggagcagggtggcaccaagttagtgaaggatctaagtcccggggaccgcgtgctggcggctgacgaccagggccggctgctgtacagcgacttcctcaccttcctggaccgcgacgaaggtgccaagaaggtcttctacgtgatcgagacgcgggagccgcgggagcgtctgctgctcactgccgcgcacctgctcttcgtggcgccgcacaacgactccgggcccactccgggaccgagcccactcttcgccagccgcgtgcgtccggggcagcgcgtgtacgtggtggctgaacgcggcggggaccgccggctgctgcccgccgcggtgcacagcgtaacgctacgagaggaggcggcgggtgcgtacgcgccgctcacggcggacggcaccattctcatcaaccgggtgctcgcctcgtgctacgcagtcatcgaggagcacagctgggcacaccgggccttcgcgcccttccgcctggcgcacgcgctgctggccgcgctggcacccgcccgcacggacggcgggggcgggggcagcatccctgccccgcaatctgtagcggaagcgaggggcgcagggccgcctgcgggcatccactggtactcgcagctgctgtaccacattggcacctggctgttggacagcgagaccctgcatcccttgggaatggcagtcaagtccagctgaagtccgacgggaccgggcagggggcgtgggggcgggcgggcgggaagcgactgccagataagcaaccgggaaagcgcacggaagga

SEQ ID NO: 6 - protein, 437 aa, Rattus norvegicus
MLLLLARCFLVALASSLLVCPGLACGPGRGFGKRQHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKARIHCSVKAENSVAAKSDGCFPGSATVHLEQGGTKLVKDLSPGDRVLAADDQGRLLYSDFLTFLDRDEGAKKVFYVIETREPRERLLLTAAHLLFVAPHNDSGPTPGPSPLFASRVRPGQRVYVVAERGGDRRLLPAAVHSVTLREEAAGAYAPLTADGTILINRVLASCYAVIEEHSWAHRAFAPFRLAHALLAALAPARTDGGGGGSIPAPQSVAEARGAGPPAGIHWYSQLLYHIGTWLLDSETLHPLGMAVKSS

SEQ ID NO: 7 - DNA, 1546 bp, Xenopus laevis
cgagcagagattgcccataattactgtctcgtctctacacccccatgtgttctgtgagcggggagctgcaccctggactttctgcacctgccttgcttgggatcggtggctagaggggtcggcgaggaggcacaaggttgctggaagcagcagcgaaggagaacatcctctgagcctttgatgtaattggcttcgctcggacgagatgctggttgcgaactcgaatctctgttggctgctgagcttcatctgcaccctggtgaccccccctgggctggcatgtggacctggccgaggcattggcaagaggagacaccccaaaaaactcacccctctcgcctataagcagttcatccccaacgtggcggagaagaccctgggggccagcggcagatacgaaggaaagattacaaggaactcggattgctttaaagaattaacccccaattataacccagatattatgtttaaagacgaggagagcaccggggcggaccggctcatgactcagagatgtaaagacaaactgaacgcactcgcgatctccgtgatgaaccagtggccgggggtgaagctgcgggtgacggaggggtgggatgaggacgggcaccacttggaggagtcgctacattatgaggggagggcagtggacatcactacgtcggaccgggaccgcagtaaatacggaatgttgggccgactggcggtggaggccgggttcgactgggtctattacgagtccaaagctcatattcactgttcggtcaaagcagagaactcagtggcggccaagtctggcgggtgcttccctgctggtgccagggtgatggtggaatttggtggcaccaaagcggtgaaagacctgcgaccaggggaccgcgttctctcctccgacccccaagggaatctgctctacagcgacttcctcatgttcatcgaccaggagcgtgacgtcaagaagctcttttacgtcatcgaaacgtctcagagaaaaattcggttgaccgcggcccatctactttttgtggcccagaccaaggtcaacggcaccaggtcgttcaagtctgtctttgccagcaacatccaaccaggagatctcatttatacagcagaatcccaagaccatgaccttgaagggcggggaaagtggagaaggttgatcttgagggaggacactggagcttatgcgcctctaactgcccatgggactgtggttatagaccaggtattggcctcctgctatgcagtcattgaggaacacacctgggcacacctcgcatttgcgccactgaggtttggcatgagcctctcctcttatatttaccccagagactccagtcctccatcaggccttcagcctcaccaccaagttgaccttcagtctcaccatcaagttgatcttcagtctcaccaccaagttgaccttcagtctcaccaccaacttgaaggcatccactggtactcccagctactgtatcagatagggacttggcttttggacagtagctccctgcacccactgggcatggcaacgaaatccagttga

SEQ ID NO: 8 - protein, 446 aa, Xenopus laevis
MLVANSNLCWLLSFICTLVTPPGLACGPGRGIGKRRHPKKLTPLAYKQFIPNVAEKTLGASGRYEGKITRNSDCFKELTPNYNPDIMFKDEESTGADRLMTQRCKDKLNALAISVMNQWPGVKLRVTEGWDEDGHHLEESLHYEGRAVDITTSDRDRSKYGMLGRLAVEAGFDWVYYESKAHIHCSVKAENSVAAKSGGCFPAGARVMVEFGGTKAVKDLRPGDRVLSSDPQGNLLYSDFLMFIDQERDVKKLFYVIETSQRKIRLTAAHLLFVAQTKVNGTRSFKSVFASNIQPGDLIYTAESQDHDLEGRGKWRRLILREDTGAYAPLTAHGTVVIDQVLASCYAVIEEHTWAHLAFAPLRFGMSLSSYIYPRDSSPPSGLQPHHQVDLQSHHQVDLQSHHQVDLQSHHQLEGIHWYSQLLYQIGTWLLDSSSLHPLGMATKSS

SEQ ID NO: 9 - DNA, 1261 bp, Homo sapiens
cccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagcaacgacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattcc

SEQ ID NO: 10 - protein, 293 aa, Homo sapiens
MNQWPGVKLRVTEGWDEDGHHSEESLHYEGRAVDITTSDRDRNKYGLLARLAVEAGFDWVYYESKAHVHCSVKSEHSAAATTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 11 - DNA, 1277 bp, Homo sapiens
ccggcgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattcccc

SEQ ID NO: 12 - protein, 312 aa, Homo sapiens
RRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 13 - DNA, 1191 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctga

SEQ ID NO: 14 - protein, 396 aa, Mus musculus [sequence omitted]